(19) United States
(12) Patent Application Publication
(10) Pub. No.: US 2023/0128412 A1
(43) Pub. Date: Apr. 27, 2023

(54) METHODS FOR ENHANCING RECOMBINANT ADENO-ASSOCIATED VIRUS YIELD

(71) Applicant: Ultragenyx Pharmaceutical Inc., Novato, CA (US)

(72) Inventors: Jan Thomas Panteli, Arlington, MA (US); Cameron Tage Fulco, West Boylston, MA (US); John Everett Allen, Reading, MA (US); Mingyang Jiang, New Haven, CT (US); Rachael C. Hogan, Everett, MA (US)

(21) Appl. No.: 17/905,866

(22) PCT Filed: Mar. 15, 2021
(86) PCT No.: PCT/US2021/022396

§ 371 Date: Sep. 8, 2022

Related U.S. Application Data
(60) Provisional application No. 62/990,099, filed on Mar. 16, 2020.

(51) Int. Cl. C12N 15/86

(52) U.S. Cl. CPC C12N 15/86; C12N 2750/14143; C12N 2750/14171; C12N 2750/14152

(57) ABSTRACT

The invention provides methods for the production of recombinant adeno-associated virus vectors (rAAV), comprising contacting a host cell with a solution comprising at least one compound of formula (I), (I-A), (I-B), (II), (III), or (IV), or a salt thereof, or a vitamin B, or any combination(s) thereof. Also provided are methods for increasing the production of rAAV by a host cell, comprising contacting a host cell with a solution comprising at least one compound of formula (I), (I-A), (I-B), (II), (III), or (IV), or a salt thereof, or a vitamin B, or any combination(s) thereof.

### CROSS-REFERENCE TO RELATED APPLICATIONS

The present application claims the benefit of and priority to U.S. Provisional Patent Application No. 62/990,099, filed Mar. 16, 2020, the entire content of which is hereby incorporated by reference in its entirety for all purposes.

### FIELD OF THE INVENTION

The invention relates generally to methods for enhancing recombinant adeno-associated virus vector (rAAV) yield, and, more particularly, the invention relates to the use of compounds to enhance rAAV yield.

### BACKGROUND

Adeno-associated virus (AAV) is a non-pathogenic, replication-defective parvovirus. Recombinant AAV vectors (rAAV) have many unique features that make them attractive as vectors for gene therapy. In particular, rAAV vectors can deliver therapeutic genes to dividing and non-dividing cells, and these genes can persist for extended periods without integrating into the genome of the targeted cell. Given the widespread therapeutic applications of rAAV, there exists an ongoing need for improved methods of rAAV vector production including methods to achieve high-titer rAAV vector yields. Previous attempts to improve the production of a variety of viral vectors have included the use of cell culture additives such as metals, trace supplements, salts, and others (see, e.g., Williams, J. Gen. Virol., 9(3): 251-5 (1970), Weinbauer et al., Limnology and Oceanography, 54(3): 774-784 (2009), Yang et al., Hepatology, 48(5): 1396-403 (2008), and U.S. Publication No. 20150353899).

### SUMMARY OF THE INVENTION

The invention is based, in part, upon the discovery that a host cell used in the production of recombinant adeno-associated virus vectors (rAAV) will produce increased amounts of rAAV, when the host cell is in contact with a solution comprising a compound described herein.

Thus, in an aspect, provided herein is a method of producing recombinant adeno-associated virus (rAAV), the method comprising contacting a host cell with a solution comprising at least one compound of formula (I) or a salt thereof:

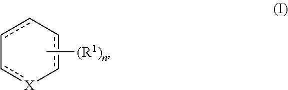

wherein:
[symbol] denotes a single bond or a double bond;
n is an integer selected from 1 to 5;
each R¹ is independently, for each occurrence, selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR;
R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and
wherein the at least one compound is not:

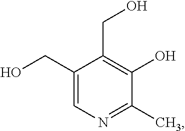 [SMILES: Cc1ncc(CO)c(CO)c1O]

or a salt thereof.

In another aspect, provided herein is a method of increasing rAAV titer yield, the method comprising contacting a host cell with a solution comprising a compound of formula (I) or a salt thereof.

In certain embodiments, the solution comprises at least one compound of formula (I-A) or a salt thereof:

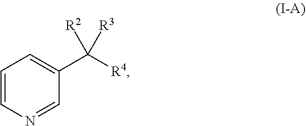

wherein:
R² and R³ are independently, for each occurrence, hydrogen or —OH,
or R² and R³ can be taken together to form oxo;
R⁴ is selected from the group consisting of hydrogen, —OH, —O—C₁₋₆alkyl, and —N(R^{e})₂; and
R^{e} is independently, for each occurrence, hydrogen or C₁₋₆alkyl.

In some embodiments, the solution comprises at least one compound selected from the group consisting of:

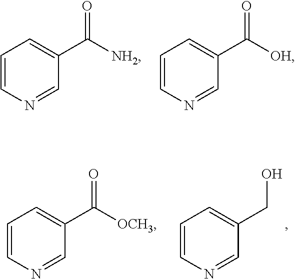

and any combination(s) thereof.

In certain embodiments, the solution comprises at least one compound of formula (I-B) or a salt thereof:

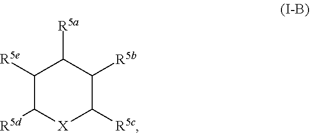

wherein:
X is CR^{c}R^{d}; and
R^{5a}, R^{5b}, R^{5c}, R^{5d}, and R^{5e} are independently, for each occurrence, —OH, —O—C₁₋₆alkyl, C₁₋₆alkyl, C₁₋₆alkyl-OH, and C₁₋₆alkyl-O—C₁₋₆alkyl; and
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{e} is selected from C₁₋₆alkyl, —OH, and —O—C₁₋₆alkyl, R^{d} is hydrogen.

In some embodiments, the solution comprises the compound of formula (III):

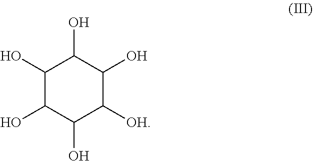 [SMILES: OC1C(O)C(O)C(O)C(O)C1O]

In another aspect, provided herein is a method of producing rAAV, the method comprising contacting a host cell with a solution comprising at least one compound of formula (II) or a salt thereof:

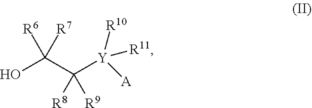

wherein:
R⁶ and R⁷ are independently, for each occurrence, selected from the group consisting of hydrogen, —OH, —CN, halogen, and C₁₋₆alkyl;
R⁸ and R⁹ are independently, for each occurrence, selected from the group consisting of hydrogen, —OH, —CN, halogen, and C₁₋₆alkyl;
Y is C or N;
R¹⁰ and R¹¹ are independently, for each occurrence, selected from the group consisting of hydrogen, —OH, and C₁₋₆alkyl;
A is selected from the group consisting of hydrogen, C₁₋₆alkyl, and —C(O)N(R^{f})₂; and
R^{f} is independently, for each occurrence, selected from the group consisting of hydrogen, C₁₋₆alkyl, C₁₋₆alkyl-C(O)OH, and C₁₋₆alkyl-OH;
wherein the at least one compound is not:

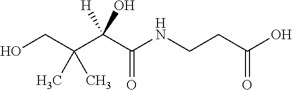

or a salt thereof.

In another aspect, provided herein is a method of increasing rAAV titer yield, the method comprising contacting a host cell with a solution comprising a compound of formula (II) or a salt thereof.

In certain embodiments, the at least one compound of formula (II) is a compound of formula (IV):

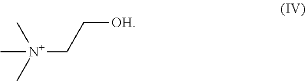

In another aspect, provided herein is a method of producing rAAV or increasing rAAV titer yield, the method comprising contacting a host cell with a solution comprising a B vitamin selected from vitamin B₂, vitamin B₇, vitamin B₉, and vitamin B₁₂.

In some embodiments, the methods as described herein may further comprise the steps of harvesting and purifying the rAAV.

It is contemplated that the concentration of a compound described herein (e.g., a compound of formula (I), (I-A), (I-B), (II), (III), or (IV), or a B vitamin (e.g., vitamin B₂, vitamin B₇, vitamin B₉, or vitamin B12)) in solution may be greater than or equal to 10 mM, greater than or equal to 12 mM, greater than or equal to 15 mM, greater than or equal to 1 mM, greater than or equal to 0.5 mM, greater than 0.5 mM, between 0.5 mM and 15 mM, or between 1 mM and 10 mM. In one embodiment, the concentration of the at least one compound (e.g., a compound of formula (I), (I-A), (I-B), (II), (III), or (IV), or a B vitamin (e.g., vitamin B₂, vitamin B₇, vitamin B₉, or vitamin B₁₂)) in the solution is sufficient to produce at least 1.2-fold greater quantities of secreted rAAV compared to that produced by a host cell not contacted with a solution comprising the at least one compound. In another embodiment, the concentration of the at least one compound (e.g., a compound of formula (I), (I-A), (I-B), (II), (III), or (IV), or a B vitamin (e.g., vitamin B₂, vitamin B₇, vitamin B₉, or vitamin B₁₂)) in solution is sufficient to produce at least 1.2-fold greater quantities of total rAAV compared to that produced by a host cell not contacted with a solution comprising the at least one compound. In one embodiment, the host cell is contacted with the solution comprising the at least one compound for at least 2 days.

It is contemplated that the host cell may be a mammalian cell, for example, a HeLa, HEK293, COS, A549, BHK, or Vero cell. It is also contemplated that the host cell may be an insect cell, for example, a Sf9, Sf-21, Tn-368, or BTI-Tn-5B1-4 (High-Five) cell. In one embodiment, the host cell is a HeLa cell. In another embodiment, the host cell is a HEK293 cell. It is contemplated that a host cell may comprise recombinant nucleic acid construct comprising a heterologous nucleotide sequence flanked by AAV inverted terminal repeats. In one embodiment, the host cell may comprise rep and cap genes (e.g., AAV rep and cap genes). In one embodiment, the host cell may comprise helper virus genes, e.g., supplied via a helper plasmid or via infection with adenovirus. In one embodiment, the host cell comprises i) a heterologous nucleotide sequence flanked by AAV inverted terminal repeats, ii) rep and cap genes, and iii) helper virus genes.

In one embodiment, the host cell produces at least 1.2-fold greater quantities of secreted rAAV compared to that produced by a host cell not contacted with a solution comprising the at least one compound (e.g., a compound of formula (I), (I-A), (I-B), (II), (III), or (IV), or a B vitamin (e.g., vitamin B₂, vitamin B₇, vitamin B₉, or vitamin B₁₂)). In another embodiment, the host cell produces at least 1.2-fold greater quantities of total rAAV compared to that produced by a host cell not contacted with a solution comprising the at least one compound (e.g., a compound of formula (I), (I-A), (I-B), (II), (III), or (IV), or a B vitamin (e.g., vitamin B₂, vitamin B₇, vitamin B₉, or vitamin B₁₂)).

In some embodiments, the invention provides methods of producing recombinant adeno-associated virus (rAAV), the method comprising contacting a host cell with a solution comprising at least one compound selected from the group consisting of:

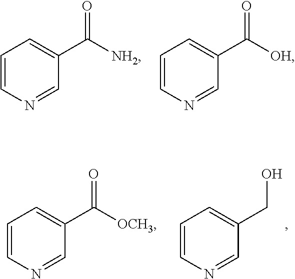

and any combination(s) thereof,
wherein the host cell comprises: i) a heterologous nucleotide sequence flanked by AAV inverted terminal repeats, ii) AAV rep and cap genes, and iii) optionally, helper virus genes.

In some embodiments, the invention provides methods of increasing rAAV titer yield, the method comprising contacting a host cell with a solution comprising at least one compound selected from the group consisting of:

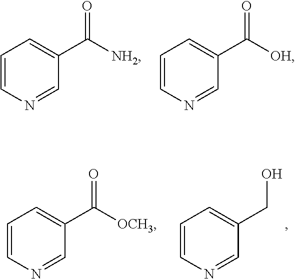

and any combination(s) thereof,
wherein the host cell comprises: i) a heterologous nucleotide sequence flanked by AAV inverted terminal repeats, ii) AAV rep and cap genes, and iii) optionally, helper virus genes.

In some embodiments, the invention provides compositions comprising a host cell and at least one compound selected from the group consisting of:

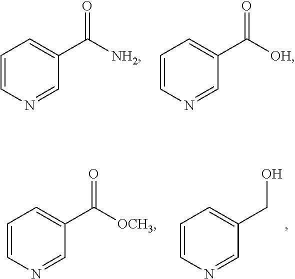

and any combination(s) thereof,
wherein the host cell comprises: i) a heterologous nucleotide sequence flanked by AAV inverted terminal repeats, ii) AAV rep and cap genes, and iii) optionally, helper virus genes.

In some embodiments, the invention provides methods of producing recombinant adeno-associated virus (rAAV), the method comprising contacting a host cell with a solution comprising

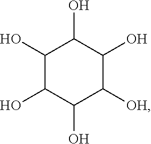 [SMILES: OC1C(O)C(O)C(O)C(O)C1O]

wherein the host cell comprises: i) a heterologous nucleotide sequence flanked by AAV inverted terminal repeats, ii) AAV rep and cap genes, and iii) optionally, helper virus genes.

In some embodiments, the invention provides methods of increasing rAAV titer yield, the method comprising contacting a host cell with a solution comprising

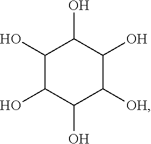 [SMILES: OC1C(O)C(O)C(O)C(O)C1O]

wherein the host cell comprises: i) a heterologous nucleotide sequence flanked by AAV inverted terminal repeats, ii) AAV rep and cap genes, and iii) optionally, helper virus genes.

In some embodiments, the invention provides compositions comprising a host cell and

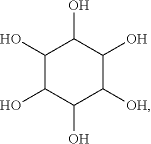 [SMILES: OC1C(O)C(O)C(O)C(O)C1O]

wherein the host cell comprises: i) a heterologous nucleotide sequence flanked by AAV inverted terminal repeats, ii) AAV rep and cap genes, and iii) optionally, helper virus genes.

In some embodiments, the invention provides methods of producing recombinant adeno-associated virus (rAAV), the method comprising contacting a host cell with a solution comprising:

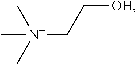

wherein the host cell comprises: i) a heterologous nucleotide sequence flanked by AAV inverted terminal repeats, ii) AAV rep and cap genes, and iii) optionally, helper virus genes.

In some embodiments, the invention provides methods of increasing rAAV titer yield, the method comprising contacting a host cell with a solution comprising:

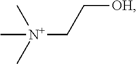

wherein the host cell comprises: i) a heterologous nucleotide sequence flanked by AAV inverted terminal repeats, ii) AAV rep and cap genes, and iii) optionally, helper virus genes.

In some embodiments, the invention provides compositions comprising a host cell and

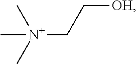

wherein the host cell comprises: i) a heterologous nucleotide sequence flanked by AAV inverted terminal repeats, ii) AAV rep and cap genes, and iii) optionally, helper virus genes.

In some embodiments, the invention provides methods of producing recombinant adeno-associated virus (rAAV), the method comprising contacting a host cell with a solution comprising at least one compound selected from the group consisting of vitamin B₂, vitamin B₇, vitamin B₉, vitamin B₁₂, and any combination(s) thereof, wherein the host cell comprises: i) a heterologous nucleotide sequence flanked by AAV inverted terminal repeats, ii) AAV rep and cap genes, and iii) optionally, helper virus genes.

In some embodiments, the invention provides methods of increasing rAAV titer yield, the method comprising contacting a host cell with a solution comprising at least one compound selected from the group consisting of vitamin B₂, vitamin B₇, vitamin B₉, vitamin B₁₂, and any combination(s) thereof, wherein the host cell comprises: i) a heterologous nucleotide sequence flanked by AAV inverted terminal repeats, ii) AAV rep and cap genes, and iii) optionally, helper virus genes.

In some embodiments of provided methods, the solution does not comprise

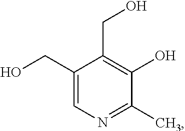 [SMILES: Cc1ncc(CO)c(CO)c1O]

or a salt thereof.

In some embodiments of provided methods, the solution does not comprise

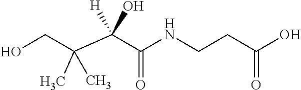

or a salt thereof.

In some embodiments, provided compositions do not comprise

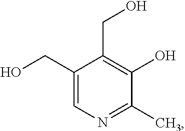 [SMILES: Cc1ncc(CO)c(CO)c1O]

or a salt thereof.

In some embodiments, provided compositions do not comprise

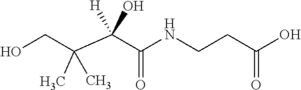

or a salt thereof.

In some embodiments, the invention provides compositions comprising a host cell and at least one compound selected from the group consisting of vitamin B₂, vitamin B₇, vitamin B₉, vitamin B₁₂, and any combination(s) thereof, wherein the host cell comprises: i) a heterologous nucleotide sequence flanked by AAV inverted terminal repeats, ii) AAV rep and cap genes, and iii) optionally, helper virus genes.

In other aspects, the invention provides a rAAV produced by any of the contemplated methods, a composition comprising a rAAV produced by any of the contemplated methods, or a composition comprising a host cell and at least one compound described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof).

These and other aspects and features of the invention are described in the following detailed description and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings.

FIG. 1 is a summary bar graph of normalized volumetric recombinant AAV (rAAV) titer for a variety of Clade E and F rAAV produced from HeLa producer cells at varying niacinamide concentrations (0.1 mM to 40 mM). Titer as measured in genome copies per mL (GC/mL) is normalized relative to rAAV production in the absence of niacinamide.

FIG. 2 is a 3^{rd}-order polynomial fit of data points of normalized volumetric rAAV titer (GC/mL) of Clade E and F rAAV produced from different HeLa producer cell clones at various niacinamide concentrations.

FIG. 3 is a bar graph depicting the effect of vessel scaling of niacinamide on rAAV production from a HeLa producer cell line capable of producing a Clade E rAAV encoding a Factor IX transgene (AAVrh10-FIX). Two common vessel scales: 3 mL-deep well (24-DW) and 100 mL shake flask (flasks) were used. Niacinamide was added at the indicated concentrations (1 mM to 50 mM) and rAAV yield (GC/mL) was measured.

FIG. 4A is a bar graph depicting the effect of niacinamide on rAAV production from a HeLa producer cell line clone capable of producing recombinant Clade E AAV encoding a Factor IX transgene (AAVrh10-FIX). Niacinamide was added at the indicated concentrations (5 mM to 40 mM) and rAAV yield (GC/mL) was measured.

FIG. 4B is a bar graph depicting the effect of niacinamide on rAAV production from a first HeLa producer cell line clone capable of producing recombinant Clade E AAV encoding a glucose 6-phosphatase (G6Pase) transgene (AAV8-G6Pase). Niacinamide was added at the indicated concentrations (5 mM to 40 mM) and rAAV yield (GC/mL) was measured.

FIG. 4C is a bar graph depicting the effect of niacinamide on rAAV production from a second HeLa producer cell line clone capable of producing recombinant Clade E AAV encoding a glucose 6-phosphatase (G6Pase) transgene (AAV8-G6Pase). Niacinamide was added at the indicated concentrations (1 mM to 40 mM) and rAAV yield (GC/mL) was measured.

FIG. 4D is a bar graph depicting the effect of niacinamide on rAAV production from a HeLa producer cell line clone capable of producing recombinant Clade E AAV encoding a Factor VIII transgene (AAVhu37-FVIII). Niacinamide was added at the indicated concentrations (1 mM to 40 mM) and rAAV yield (GC/mL) was measured.

FIG. 5 is a bar graph depicting the effect of niacinamide on rAAV production from two HeLa producer cell line clones capable of producing recombinant Clade F AAV encoding a truncated ATP7B transgene (AAV9-ATP7B). The clones were cultured in a 2 L bioreactor. Niacinamide was added at the indicated concentration (3 mM) and rAAV yield (GC/mL) was measured.

FIG. 6 is a bar graph depicting the effect of niacinamide on rAAV production from HEK293 cells cultured in 30 mL flasks. The HEK293 cells were transfected with plasmids enabling the production of recombinant Clade E AAV encoding an ornithine transcarbamylase (rAAV8-OTC). Niacinamide was added at the indicated concentrations (0.03 mM to 30 mM) and rAAV yield (GC/mL) was measured.

### DETAILED DESCRIPTION

The invention is based, in part, upon the discovery that a host cell used in the production of recombinant adeno-associated virus vectors (rAAV) will produce increased amounts of rAAV when a compound, such as a compound of formula (I), a compound of formula (I-A), a compound of formula (I-B), a compound of formula (II), a compound of formula (III), a compound of formula (IV), or a salt of any one thereof, or a B vitamin, is added to the host cell culture.

### I. Compounds Used in the Methods

In one aspect, the invention provides a method of producing recombinant adeno-associated virus (rAAV), the method comprising contacting a host cell with a solution comprising at least one compound of formula (I):

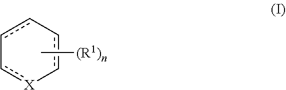

or a salt thereof, wherein:
   [symbol] denotes a single bond or a double bond;
   n is an integer selected from 1 to 5;
   each R¹ is independently, for each occurrence, selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b};
   R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and
wherein the at least one compound is not:

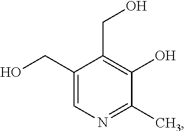 [SMILES: Cc1ncc(CO)c(CO)c1O]

or a salt thereof.

In another aspect, the invention provides a method of increasing rAAV titer yield, the method comprising contacting a host cell with a solution comprising a compound of formula (I) or a salt thereof.

In certain embodiments, a compound of formula (I) described herein is a compound in which X is N. In some embodiments, a compound of formula (I) described herein is a compound in which X is N and R¹ is independently, for each occurrence, selected from —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b}. In certain embodiments, a compound of formula (I) described herein is a compound in which X is N and R¹ is independently, for each occurrence, selected from C₁₋₆alkyl-OH, —C(O)N(R^{a})₂, and —C(O)OR. In certain embodiments, a compound of formula (I) described herein is a compound in which X is N and R¹ is C₁₋₆alkyl-OH (for example, —CH₂OH). In certain embodiments, a compound of formula (I) described herein is a compound in which X is N; R¹ is —C(O)N(R^{a})₂; and R^{a} is hydrogen (for example, —C(O)NH₂). In certain embodiments, a compound of formula (I) described herein is a compound in which X is N; R¹ is —C(O)OR^{b}; and R^{b} is independently, for each occurrence, hydrogen or methyl (for example, C(O)OH and —C(O)OCH₃).

In certain embodiments, a compound of formula (I) described herein is a compound in which X is CR^{c}R^{d}; R^{c} is hydrogen; R^{d} is selected from C₁₋₆alkyl, —OH, and —O—C₁₋₆alkyl; and each R¹ is independently, for each occurrence, selected from —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR. In some embodiments, a compound of formula (I) described herein is a compound in which X is CR^{c}R^{d}; R^{c} is selected from C₁₋₆alkyl, —OH, and —O—C₁₋₆alkyl; R^{d} is —OH; and R¹ is independently, for each occurrence, selected from —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b}. In certain embodiments, a compound of formula (I) described herein is a compound in which X is CR^{c}R^{d}; R^{c} is hydrogen; R^{d} is —OH; and R¹ is independently, for each occurrence, selected from —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR. In certain embodiments, a compound of formula (I) described herein is a compound in which X is CR^{c}R^{d}; R^{c} is hydrogen; R^{d} is —OH; and R¹ is —OH.

In certain embodiments, a compound of formula (I) described herein is a compound in which X is N; n is 1; and R¹ is independently, for each occurrence, selected from C₁₋₆alkyl-OH, —C(O)N(R^{a})₂, and —C(O)OR (for example, R¹ is independently, for each occurrence, selected from —CH₂OH, —C(O)NH₂, —C(O)OH, and —C(O)OCH₃). In certain embodiments, a compound of formula (I) described herein is a compound in which X is CR^{c}R^{d}; R^{c} is hydrogen; R^{d} is —OH; n is 5; and R¹ is —OH.

In certain embodiments, the invention provides a method of producing recombinant adeno-associated virus (rAAV), the method comprising contacting a host cell with a solution comprising at least one compound of formula (I-A) or a salt thereof. In certain embodiments, the invention provides a method of increasing rAAV titer yield, the method comprising contacting a host cell with a solution comprising a compound of formula (I-A) or a salt thereof:

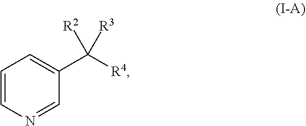

wherein:
R² and R³ are independently, for each occurrence, hydrogen or —OH,
or R² and R³ can be taken together to form oxo;
R⁴ is selected from the group consisting of hydrogen, —OH, —O—C₁₋₆alkyl, and —N(R^{e})₂; and
R^{e} is independently, for each occurrence, hydrogen or C₁₋₆alkyl.

In certain embodiments, a compound of formula (I-A) described herein is a compound in which R² and R³ are taken together to form oxo. In certain embodiments, a compound of formula (I-A) is a compound in which R⁴ is selected from —OH, —O—C₁₋₆alkyl, and —N(R^{e})₂ (for example, —NH₂, —OH, and —OCH₃). In certain embodiments, a compound of formula (I-A) described herein is a compound in which R⁴ is selected from —NH₂, —OH, and —OCH₃. In certain embodiments, a compound of formula (I-A) described herein is a compound in which R² and R³ are taken together to form oxo, and R⁴ is selected from —OH, —O—C₁₋₆alkyl, and —N(R^{e})₂ (for example, —NH₂, —OH, and —OCH₃). In some embodiments, a compound of formula (I-A) described herein is a compound in which R² and R³ are taken together to form oxo, and R⁴ is selected from —NH₂, —OH, and —OCH₃. In certain embodiments, a compound of formula (I-A) described herein is a compound in which R² and R³ are taken together to form oxo, and R⁴ is —NH₂. In certain embodiments, a compound of formula (I-A) described herein is a compound in which R² and R³ are taken together to form oxo, and R⁴ is —OH. In certain embodiments, a compound of formula (I-A) described herein is a compound in which R² and R³ are taken together to form oxo, and R⁴ is —OCH₃.

In certain embodiments, a compound of formula (I-A) described herein is a compound in which R² and R³ are both hydrogen. In certain embodiments, a compound of formula (I-A) is a compound in which R⁴ is selected from hydrogen, —OH, —O—C₁₋₆alkyl, and —N(R^{e})₂ (for example, —NH₂, —OH, and —OCH₃). In certain embodiments, a compound of formula (I-A) described herein is a compound in which R⁴ is selected from hydrogen, —NH₂, —OH, and —OCH₃. In certain embodiments, a compound of formula (I-A) described herein is a compound in which R² and R³ are both hydrogen, and R⁴ is selected from the group consisting of hydrogen, —NH₂, —OH, and —OCH₃. In certain embodiments, a compound of formula (I-A) described herein is a compound in which R² and R³ are both hydrogen, and R⁴ is hydrogen. In certain embodiments, a compound of formula (I-A) described herein is a compound in which R² and R³ are both hydrogen, and R⁴ is —NH₂. In certain embodiments, a compound of formula (I-A) described herein is a compound in which R² and R³ are both hydrogen, and R⁴ is —OH. In certain embodiments, a compound of formula (I-A) described herein is a compound in which R² and R³ are both hydrogen, and R⁴ is —OCH₃.

In certain embodiments, a compound of formula (I-A) described herein is a compound in which R² and R³ are independently, for each occurrence, hydrogen or —OH, or R² and R³ can be taken together to form oxo; and R⁴ is —OH. In certain embodiments, a compound of formula (I-A) described herein is a compound in which R² and R³ are independently, for each occurrence, hydrogen or —OH, or R² and R³ can be taken together to form oxo; and R⁴ is —OCH₃. In certain embodiments, a compound of formula (I-A) described herein is a compound in which R² and R³ are independently, for each occurrence, hydrogen or —OH, or R² and R³ can be taken together to form oxo; and R⁴ is —NH₂.

In certain embodiments, the invention provides a method of producing rAAV, the method comprising contacting a host cell with a solution comprising at least one compound selected from the group consisting of:

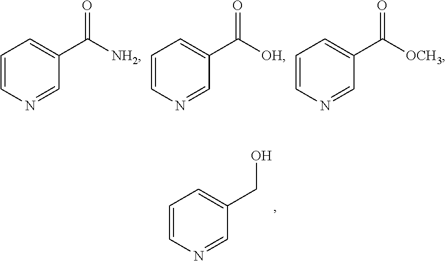

and any combination(s) thereof.

In certain embodiments, provided are methods of producing rAAV, the method comprising contacting a host cell with a solution comprising at least one compound selected from the group consisting of: niacinamide, niacin, methyl-nicotinate, nicotinyl alcohol, and any combination(s) thereof.

In certain embodiments, the invention provides a method of increasing rAAV titer yield, the method comprising contacting a host cell with a solution comprising at least one compound selected from the group consisting of:

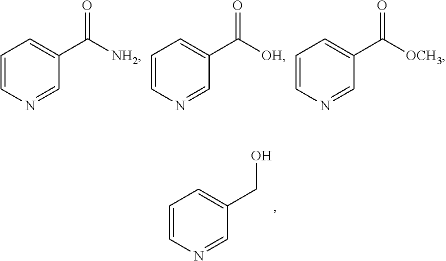

and any combination(s) thereof.

In certain embodiments, provided are methods of increasing rAAV titer yield, the method comprising contacting a host cell with a solution comprising at least one compound selected from the group consisting of: niacinamide, niacin, methyl-nicotinate, nicotinyl alcohol, and any combination(s) thereof.

In certain embodiments, the invention provides a method of producing rAAV, the method comprising contacting a host cell with a solution comprising at least one compound of formula (I-B) or a salt thereof. In some embodiments, the invention provides a method of increasing rAAV titer yield, the method comprising contacting a host cell with a solution comprising at least one compound of formula (I-B) or a salt thereof:

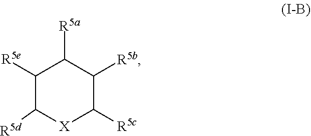

wherein:
X is CR^{c}R^{d}; and
R^{5a}, R^{5b}, R^{5c}, R^{5d}, and R^{5e} are independently, for each occurrence, —OH, —O—C₁₋₆alkyl, C₁₋₆alkyl, C₁₋₆alkyl-OH, and C₁₋₆alkyl-O—C₁₋₆alkyl,
wherein R^{c} and R^{d} are independently, for each occurrence, selected from hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{e} is selected from C₁₋₆alkyl, —OH, and —O—C₁₋₆alkyl, R^{d} is hydrogen.

In certain embodiments, a compound of formula (I-B) described herein is a compound in which R^{c} is hydrogen, and R^{d} is selected from C₁₋₆alkyl, —OH, and —O—C₁₋₆alkyl. In certain embodiments, a compound of formula (I-B) described herein is a compound in which R^{e} is selected from C₁₋₆alkyl, —OH, and —O—C₁₋₆alkyl, and R^{d} is hydrogen. In some embodiments, a compound of formula (I-B) described herein is a compound in which R^{c} is hydrogen and R^{d} is —OH. In certain embodiments, a compound of formula (I-B) described herein is a compound in which R^{c} is hydrogen and R^{d} is —O—C₁₋₆alkyl. In certain embodiments, a compound of formula (I-B) described herein is a compound in which R^{c} is hydrogen and R^{d} is C₁₋₆alkyl.

In certain embodiments, a compound of formula (I-B) described herein is a compound in which R^{5a}, R^{5b}, R^{5c}, R^{5d}, and R^{5e} are —OH. In certain embodiments, a compound of formula (I-B) described herein is a compound in which R^{c} is hydrogen and R^{d} is —OH. In certain embodiments, a compound of formula (I-B) described herein is a compound in which R^{5a}, R^{5b}, R^{5c}, R^{5d}, and R^{5e} are —OH, and R^{c} and R^{d} are independently, for each occurrence, selected from hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{e} is selected from C₁₋₆alkyl, —OH, and —O—C₁₋₆alkyl, R^{d} is hydrogen. In certain embodiments, a compound of formula (I-B) described herein is a compound in which R^{5a}, R^{5b}, R^{5c}, R^{5d}, and R^{5e} are —OH, R^{c} is hydrogen, and R^{d} is selected from C₁₋₆alkyl, —OH, and —O—C₁₋₆alkyl. In certain embodiments, a compound of formula (I-B) described herein is a compound in which R^{5a}, R^{5b}, R^{5c}, R^{5d}, and R^{5e} are —OH, R^{c} is hydrogen, and R^{d} is selected from —OH.

In certain embodiments, the invention provides a method of producing rAAV, the method comprising contacting a host cell with a solution comprising a compound of formula (III) or a salt thereof:

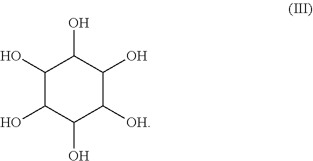 [SMILES: OC1C(O)C(O)C(O)C(O)C1O]

In certain embodiments, the invention provides a method of producing rAAV, the method comprising contacting a host cell with a solution comprising myo-inositol or a salt thereof.

In certain embodiments, the invention provides a method of increasing rAAV titer yield, the method comprising contacting a host cell with a solution comprising a compound of formula (III) or a salt thereof:

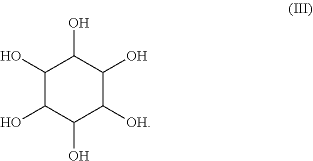 [SMILES: OC1C(O)C(O)C(O)C(O)C1O]

In certain embodiments, the invention provides a method of increasing rAAV titer yield, the method comprising contacting a host cell with a solution comprising myo-inositol or a salt thereof.

In another aspect, the invention provides a method of producing rAAV, the method comprising contacting a host cell with a solution comprising a compound of formula (II) or a salt thereof. In another aspect, the invention provides a method of increasing rAAV titer yield, the method comprising contacting a host cell with a solution comprising at least one compound of formula (II) or a salt thereof:

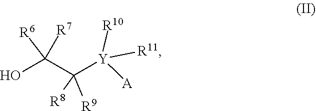

wherein:
   R⁶ and R⁷ are independently, for each occurrence, selected from hydrogen, —OH, —CN, halogen, and C₁₋₆alkyl;
   R⁸ and R⁹ are independently, for each occurrence, selected from hydrogen, —OH, —CN, halogen, and C₁₋₆alkyl;
   Y is C or N;
   R¹⁰ and R¹¹ are independently, for each occurrence, selected from hydrogen, —OH, and C₁₋₆alkyl;
   A is selected from the group consisting of hydrogen, C₁₋₆alkyl, and —C(O)N(R^{f})₂; and
   R^{f} is independently, for each occurrence, selected from hydrogen, C₁₋₆alkyl, C₁₋₆alkyl-C(O)OH, and C₁₋₆alkyl-OH;
wherein the at least one compound is not:

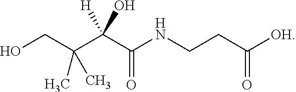

In certain embodiments, a compound of formula (II) described herein is a compound in which R⁶ and R⁷ are hydrogen. In certain embodiments, a compound of formula (II) described herein is a compound in which R⁸ and R⁹ are hydrogen. In certain embodiments, a compound of formula (II) is a compound in which R⁶ and R⁷ are hydrogen, and R⁸ and R⁹ are independently, for each occurrence, selected from hydrogen, —OH, —CN, halogen, and C₁₋₆alkyl. In certain embodiments, a compound of formula (II) described herein is a compound in which R¹ and R⁹ are hydrogen, and R⁶ and R⁷ are independently, for each occurrence, selected from hydrogen, —OH, —CN, halogen, and C₁₋₆alkyl. In certain embodiments, a compound of formula (II) described herein is a compound in which R⁶, R⁷, R⁸, and R⁹ are hydrogen.

In certain embodiments, a compound of formula (II) described herein is a compound in which Y is C. In certain embodiments, a compound of formula (II) described herein is a compound in which Y is C; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is C; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is C and R⁶, R⁷, R⁸, and R⁹ are hydrogen.

In certain embodiments, a compound of formula (II) described herein is a compound in which Y is N. In certain embodiments, a compound of formula (II) described herein is a compound in which Y is N; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is N; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is N and R⁶, R⁷, R⁸, and R⁹ are hydrogen.

In certain embodiments, a compound of formula (II) described herein is a compound in which R¹⁰ and R¹¹ are C₁₋₆alkyl. In certain embodiments, a compound of formula (II) described herein is a compound in which R¹⁰ and R¹¹ are CH₃.

In certain embodiments, a compound of formula (II) described herein is a compound in which Y is C; R¹⁰ and R¹¹ are C₁₋₆alkyl; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is C; R¹⁰ and R¹¹ are C₁₋₆alkyl; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is C; R¹⁰ and R¹¹ are C₁₋₆alkyl; and R⁶, R⁷, R⁸, and R⁹ are hydrogen.

In certain embodiments, a compound of formula (II) described herein is a compound in which Y is N; R¹⁰ and R¹¹ are C₁₋₆alkyl; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is N; R¹⁰ and R¹¹ are C₁₋₆alkyl; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is N; R¹⁰ and R¹¹ are C₁₋₆alkyl; and R⁶, R⁷, R⁸, and R⁹ are hydrogen.

In certain embodiments, a compound of formula (II) described herein is a compound in which Y is C; R¹⁰ and R¹¹ are CH₃; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is C; R¹⁰ and R¹¹ are CH₃; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is C; R¹⁰ and R¹¹ are CH₃; and R⁶, R⁷, R⁸, and R⁹ are hydrogen.

In certain embodiments, a compound of formula (II) described herein is a compound in which Y is N; R¹⁰ and R¹¹ are CH₃; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is N; R¹⁰ and R¹¹ are CH₃; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is N; R¹⁰ and R¹¹ are CH₃; and R⁶, R⁷, R⁸, and R⁹ are hydrogen.

In certain embodiments, a compound of formula (II) described herein is a compound in which A is C₁₋₆alkyl. In certain embodiments, a compound of formula (II) described herein is a compound in which A is CH₃.

In certain embodiments, a compound of formula (II) described herein is a compound in which Y is C; A is C₁₋₆alkyl; R¹⁰ and R¹¹ are CH₃; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is C; A is C₁₋₆alkyl; R¹⁰ and R¹¹ are CH₃; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is C; A is C₁₋₆alkyl; R¹⁰ and R¹¹ are CH₃; and R⁶, R⁷, R⁸, and R⁹ are hydrogen.

In certain embodiments, a compound of formula (II) described herein is a compound in which Y is N; A is C₁₋₆alkyl; R¹⁰ and R¹¹ are CH₃; R⁶ and R⁷ are hydrogen; and R⁸ and R⁹ are independently, for each occurrence, selected from hydrogen, —OH, —CN, halogen, and C₁₋₆alkyl. In certain embodiments, a compound of formula (II) described herein is a compound in which Y is N; A is C₁₋₆alkyl; R¹⁰ and R¹¹ are CH₃; R⁸ and R⁹ are hydrogen; and R⁶ and R⁷ are independently, for each occurrence, selected from hydrogen, —OH, —CN, halogen, and C₁₋₆alkyl. In certain embodiments, a compound of formula (II) described herein is a compound in which Y is N; A is C₁₋₆alkyl; R¹⁰ and R¹¹ are CH₃; and R⁶, R⁷, R⁸, and R⁹ are hydrogen.

In certain embodiments, a compound of formula (II) described herein is a compound in which R⁶, R⁷, R⁸, and R⁹ are hydrogen, Y is N, R¹⁰ and R¹¹ are CH₃, and A is CH₃.

In certain embodiments, the invention provides a method of producing rAAV, the method comprising contacting a host cell with a solution comprising a compound of formula (IV) or a salt thereof:

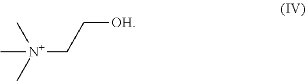

In certain embodiments, the invention provides a method of producing rAAV, the method comprising contacting a host cell with a solution comprising choline or a salt thereof.

In certain embodiments, the invention provides a method of increasing rAAV titer yield, the method comprising contacting a host cell with a solution comprising a compound of formula (IV) or a salt thereof:

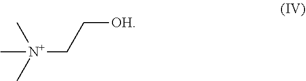

In certain embodiments, the invention provides a method of increasing rAAV titer yield, the method comprising contacting a host cell with a solution comprising choline or a salt thereof.

In another aspect, the invention provides a method of producing rAAV, the method comprising contacting a host cell with a solution comprising at least one B vitamin selected from vitamin B₂, vitamin B₇, vitamin B₉, vitamin B₁₂, or any combination(s) thereof.

In another aspect, the invention provides a method of increasing rAAV titer yield, the method comprising contacting a host cell with a solution comprising at least one B vitamin selected from vitamin B₂, vitamin B₇, vitamin B₉, vitamin B₁₂, or any combination(s) thereof.

### II. Culture Conditions

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is greater than 0.5 mM. In other embodiments, the final concentration of the at least one compound in the solution is greater than or equal to 0.5 mM.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is between 0.5 mM and 15 mM (e.g., between 0.5 mM and 14 mM, between 0.5 mM and 13 mM, between 0.5 mM and 12 mM, between 0.5 mM and 11 mM, between 0.5 mM and 10 mM, between 0.5 mM and 9 mM, between 0.5 mM and 8 mM, between 0.5 mM and 7 mM, between 0.5 mM and 6 mM, between 0.5 mM and 5 mM, between 0.5 mM and 4 mM, between 0.5 mM and 3 mM, between 0.5 mM and 2 mM, between 0.5 mM and 1 mM, between 1 mM and 15 mM, between 2 mM and 15 mM, between 3 mM and 15 mM, between 4 mM and 15 mM, between 5 mM and 15 mM, between 6 mM and 15 mM, between 7 mM and 15 mM, between 8 mM and 15 mM, between 9 mM and 15 mM, between 10 mM and 15 mM, between 11 mM and 15 mM, between 12 mM and 15 mM, between 13 mM and 15 mM, and between 14 mM and 15 mM). In some exemplary embodiments, the final concentration of the at least one compound in the solution is between 1 mM and 10 mM. In some embodiments, the final concentration of the at least one compound in the solution is selected from 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, or 15 mM, and all decimal and fractional values in between 0.5 mM and 15 mM.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is sufficient to produce at least 1.2-fold greater quantities of secreted rAAV compared to that produced by a host cell not contacted with a solution comprising the at least one compound. In some embodiments, the increase may be 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, or 5-fold, and all decimal and fractional values in between 1.2-fold and 5-fold.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is sufficient to produce at least 1.2-fold greater quantities of total rAAV compared to that produced by a host cell not contacted with a solution comprising the at least one compound. In some embodiments, the increase may be 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, or 5-fold, and all decimal and fractional values in between 1.2-fold and 5-fold.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is sufficient to produce 1.2 to 2.5-fold greater quantities of secreted rAAV compared to that produced by a host cell not contacted with a solution comprising the at least one compound.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, vitamin B₁₂, or any combination(s) thereof), or any combination(s) thereof), wherein the final concentration of the at least one compound in the solution is sufficient to produce 1.2 to 2.5-fold greater quantities of total rAAV compared to that produced by a host cell not contacted with a solution comprising the at least one compound.

In certain embodiments, the host cell is contacted with the solution comprising the at least one compound for at least 2 days.

In certain embodiments, the methods further comprise the steps of harvesting and purifying the rAAV.

In certain embodiments, the host cell is a mammalian cell. In some embodiments, the host cell is selected from HeLa, HEK293, COS, A549, BHK, and Vero cells.

In one embodiment, the host cell is a HeLa cell. It will be understood and readily appreciated by the skilled artisan that, included within the meaning of HeLa cell, is any clonal derivative, e.g., a HeLa S3 cell, which is a subclone of the HeLa cell line that can grow in serum-free medium as well as suspension cultures.

In another embodiment, the host cell is a HEK293 cell. It will be understood and readily appreciated by the skilled artisan that, included with the meaning of HEK293 cell, is any clonal derivative, e.g., a HEK293-F cell, HEK293-T cell, or a HEK-EXP1293 cell.

In certain embodiments, the host cell is an insect cell. In some embodiments, the host cell is selected from the group consisting of Sf9, Sf-21, Tn-368, and BTI-Tn-5B1-4 (High-Five) cells.

In some embodiments, the host cell comprises a heterologous nucleotide sequence flanked by AAV inverted terminal repeats. In some embodiments, the host cell comprises rep and cap genes (e.g., AAV rep and cap genes). In some embodiments, the host cell comprises helper virus genes. In certain embodiments, the host cell comprises i) a heterologous nucleotide sequence flanked by AAV inverted terminal repeats, ii) rep and cap genes (e.g., AAV rep and cap genes), and iii) helper virus genes.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, and vitamin B₁₂, or any combination(s) thereof), or any combination(s) thereof), wherein the host cell produces at least 1.2-fold greater quantities of secreted rAAV compared to that produced by a host cell not contacted with a solution comprising the at least one compound.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, and vitamin B₁₂, or any combination(s) thereof), or any combination(s) thereof), wherein the host cell produces at least 1.2-fold greater quantities of total rAAV compared to that produced by a host cell not contacted with a solution comprising the at least one compound.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, and vitamin B₁₂, or any combination(s) thereof), or any combination(s) thereof), wherein the host cell produces 1.2 to 2.5-fold greater quantities of secreted rAAV compared to that produced by a host cell not contacted with a solution comprising the at least one compound.

In certain embodiments, the invention provides a method of producing rAAV or increasing rAAV titer yield, the method comprising contacting a host cell with a solution comprising at least one compound as described herein (e.g., a compound of formula (I), (I-A), (I-B), (II), (III), or (IV), or a B vitamin (e.g., vitamin B₂, vitamin B₇, vitamin B₉, and vitamin B₁₂, or any combination(s) thereof), or any combination(s) thereof), wherein the host cell produces 1.2 to 2.5-fold greater quantities of total rAAV compared to that produced by a host cell not contacted with a solution comprising the at least one compound.

In certain embodiments, the solution comprises cell culture medium. In some embodiments, the solution comprises production medium.

In certain embodiments, the methods comprise culturing the host cell in a suspension culture. In certain embodiments, the methods comprise culturing the host cell in an adherent culture. In certain embodiments, the methods comprise culturing the host cell in a 1 L bioreactor. In certain embodiments, the methods comprise culturing the host cell in a 2 L bioreactor. In certain embodiments, the methods comprise culturing the host cell in a 3 L bioreactor. In certain embodiments, the methods comprise culturing the host cell in a 250 L bioreactor. In certain embodiments, the methods comprise culturing the host cell in a 500 L bioreactor. In certain embodiments, the methods comprise culturing the host cell in a 2,000 L bioreactor.

### III. Compositions

In an aspect, the invention provides a composition comprising a host cell and a compound of formula (I) or a salt thereof.

In another aspect, the invention provides a composition comprising a host cell and a compound of formula (I-A) or a salt thereof.

In some embodiments, the invention provides a composition comprising a host cell and at least one compound selected from the group consisting of:

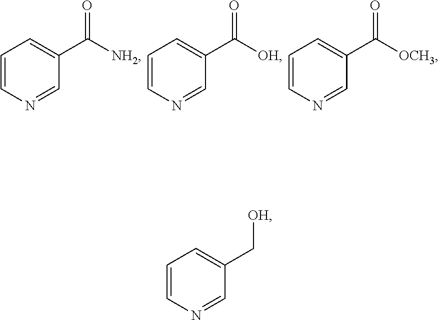

and any combination(s) thereof.

In some embodiments, the invention provides a composition comprising a host cell and at least one compound selected from the group consisting of niacinamide, niacin, methyl-nicotinate, nicotinyl alcohol, and any combination(s) thereof.

In an aspect, the invention provides a composition comprising a host cell and at least one compound of formula (I-B) or a salt thereof.

In certain embodiments, the invention provides a composition comprising a host cell and a compound of formula (III) or a salt thereof:

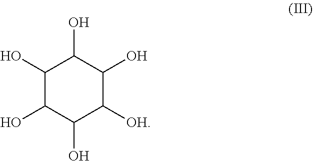 [SMILES: OC1C(O)C(O)C(O)C(O)C1O]

In certain embodiments, the invention provides a composition comprising a host cell and myo-inositol or a salt thereof.

In another aspect, the invention provides a composition comprising a host cell and a compound of formula (II) or a salt thereof.

In certain embodiments, the invention provides a composition comprising a host cell and a compound of formula (IV) or a salt thereof:

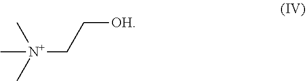

In certain embodiments, the invention provides a composition comprising a host cell and a compound of choline or a salt thereof.

In an aspect, the invention provides a composition comprising a host cell and at least one B vitamin selected from vitamin B₂, vitamin B₇, vitamin B₉, vitamin B₁₂, or any combination(s) thereof.

### IV. Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₅ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Oxo group” refers to —C(═O)—.

“Halo” or “halogen” refers to fluoro (F), chloro (C₁), bromo (Br), and iodo (I). In certain embodiments, the halogen group is either fluoro or chloro.

A “counterion” or “anionic counterion” is a negatively charged group associated with a cationic quaternary amino group in order to maintain electronic neutrality. Exemplary counterions include halide ions (e.g., F—, Cl—, Br—, I—), NO3-, ClO4-, OH—, H2PO4-, HSO4-, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J Pharm. Sci. (1977) 66(1): 1-79.

These and other exemplary substituents are described in more detail in the Detailed Description, and Claims. The invention is not intended to be limited in any manner by the above exemplary listing of substituents.

### V. Adeno-Associated Virus

Adeno-associated virus (AAV) is a small, nonenveloped icosahedral virus of the genus Dependoparvovirus and family Parvovirus. AAV has a single-stranded linear DNA genome of approximately 4.7 kb. AAV includes numerous serologically distinguishable types including serotypes AAV-1 to AAV-12, as well as more than 100 serotypes from nonhuman primates (See, e.g., Srivastava, J. Cell Biochem., 105(1): 17-24 (2008), and Gao et al., J. Virol., 78(12), 6381-6388 (2004)). Any AAV type may be used in the methods of the present invention. AAV is capable of infecting both dividing and quiescent cells of several tissue types, with different AAV serotypes exhibiting different tissue tropism. AAV is non-autonomously replicating, and has a life cycle with a latent phase and an infectious phase. In the latent phase, after a cell is infected with an AAV, the AAV site-specifically integrates into the host's genome as a provirus. The infectious phase does not occur unless the cell is also infected with a helper virus (for example, adenovirus (AV) or herpes simplex virus), which allows the AAV to replicate.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous protein of interest. In this case the rep and cap proteins (e.g., AAV rep and cap proteins) are provided in trans on, for example, a plasmid. In order to produce an AAV vector, a host cell line permissive of AAV replication typically should express the rep and cap genes, the ITR-flanked expression cassette, and helper functions provided by a helper virus, for example adenovirus (AV) genes E1a, E1b55K, E2a, E4orf6, and VA (Weitzman et al., Adeno-associated virus biology. Adeno-Associated Virus: Methods and Protocols, pp. 1-23, 2011). Production of AAV vector can also result in the production of helper virus particles, which, in some embodiments, must be removed or inactivated prior to use of the AAV vector. Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, 8,163,543, U.S. Publication No. 20020081721, PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes. In certain embodiments, AAV vectors are produced in these cell types by one plasmid containing the ITR-flanked expression cassette and AAV virus genes and one or more additional plasmids providing the helper virus genes.

AAV of any serotype may be used in the present invention. Similarly, it is contemplated that any AV type may be used, and a person of skill in the art will be able to identify AAV and AV types suitable for the production of their desired recombinant AAV vector (rAAV). AAV and AV particles may be purified, for example by affinity chromatography, iodixanol gradient, or CsCl gradient.

The genome of wild-type AAV is single-stranded DNA and is 4.7 kb. AAV vectors may have single-stranded genomes that are 4.7 kb in size, or are larger or smaller than 4.7 kb, including oversized genomes that are as large as 5.2 kb, or as small as 3.0 kb. Further, vector genomes may be substantially self-complementary, so that within the virus the genome is substantially double stranded. AAV vectors containing genomes of all types are suitable for use in the method of the instant invention.

As discussed above, AAV requires co-infection with a helper virus in order to enter the infectious phase of its life cycle. Helper viruses include Adenovirus (AV), and herpes simplex virus (HSV), and systems exist for producing AAV in insect cells using baculovirus. It has also been proposed that papilloma viruses may also provide a helper function for AAV (See, e.g., Hermonat et al., Molecular Therapy 9, 5289-5290 (2004)). Helper viruses include any virus capable of creating an allowing AAV replication. AV is a nonenveloped nuclear DNA virus with a double-stranded DNA genome of approximately 36 kb. AV is capable of rescuing latent AAV provirus in a cell, by providing E1a, E1b55K, E2a, E4orf6, and VA genes, allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as typically necessary for HSV-1 helper functions for AAV replication: the helicase/primase complex (ULS, UL8, and UL52) and the DNA binding protein ICP8 encoded by the UL29 gene, with other proteins enhancing the helper function.

### VI. Production of rAAV

The present invention comprises the production of a recombinant adeno-associated virus vector (rAAV) from a host cell, using any suitable method known in the art. As used herein, the term “host cell” refers to any cell or cells capable of producing a rAAV. In some embodiments, the host cell is a mammalian cell, for example, a HeLa cell, COS cell, HEK293 cell, A549 cell, BHK cell, or Vero cell. In other embodiments, the host cell is an insect cell, for example, a Sf9 cell, Sf-21 cell, Tn-368 cell, or BTI-Tn-5B1-4 (High-Five) cell. Unless otherwise indicated, the terms “cell” or “cell line” are understood to include modified or engineered variants of the indicated cell or cell line.

As discussed above, to allow for production of rAAV, the host cell typically should be provided with AAV inverted terminal repeats (ITRs) (which may, for example, flank a heterologous nucleotide sequence of interest), AAV rep and cap gene functions, and, additional helper functions. These may be provided to the host cell using any number of appropriate plasmids or vectors. Additional helper functions can be provided by, for example, an adenovirus (AV) infection, by a plasmid that carries all of the required AV helper function genes, or by other viruses such as HSV or baculovirus. Any genes, gene functions, or other genetic material necessary for rAAV production by the host cell may, for example, transiently exist within the host cell, or be stably inserted into the host cell genome. In some embodiments, the host cell is a producer cell comprising AAV rep and cap gene functions and a rAAV vector genome. In some embodiments, the host cell is a packaging cell comprising AAV rep and cap gene functions which at the time of production is provided a rAAV vector genome by a separate recombinant virus. rAAV production methods suitable for use with the methods of the current invention include those disclosed in Clark et al., Human Gene Therapy 6:1329-1341 (1995), Martin et al., Human Gene Therapy Methods 24:253-269 (2013), Thorne et al., Human Gene Therapy 20:707-714 (2009), Fraser Wright, Human Gene Therapy 20:698-706 (2009), and Virag et al., Human Gene Therapy 20:807-817 (2009).

### VI. Purification of rAAV Particles

In some embodiments of the current invention, the rAAV particles are harvested and/or purified from the host cell after the host cell has been contacted with the solution comprising a compound described herein (e.g., a compound of formula (I), (I-A), (I-B), (II), (III), or (IV), or a B vitamin (e.g., vitamin B₂, vitamin B₇, vitamin B₉, and vitamin B₁₂)), or a salt thereof rAAV particles may be obtained from host cells by lysing the cells. Lysis of host cells can be accomplished by methods that chemically or enzymatically treat the cells in order to release infections viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used. Alternatively, supernatant may be collected from host cells without the need for cell lysis. As used herein, “total rAAV” refers to the total rAAV produced by a host cell, and “secreted rAAV” refers to rAAV that can be can be harvested from a host cell without the need to cell lysis.

After harvesting rAAV particles, it may be desired to purify the sample containing rAAV, to remove, for example, the cellular debris resulting from cell lysis. Methods of minimal purification of AAV particles are known in the art. Two exemplary purification methods are Cesium chloride (CsCl)— and iodixanol-based density gradient purification. Both methods are described in Strobel et al., Human Gene Therapy Methods, 26(4): 147-157 (2015). Minimal purification can also be accomplished using affinity chromatography using, for example AVB Sepharose affinity resin (GE Healthcare Bio-Sciences AB, Uppsala, Sweden). Methods of AAV purification using AVB Sepharose affinity resin are described in, for example, Wang et al., Mol Ther Methods Clin Dev., 2:15040 (2015). Following purification, rAAV particles may be filtered and stored at ≤−60° C.

### VII. Quantification of rAAV Particles

Quantification of rAAV particles is complicated by the fact that AAV infection does not result in cytopathic effect in vitro, and therefore plaque assays cannot be used to determine infectious titers. rAAV particles can be quantified using a number of methods, however, including quantitative polymerase chain reaction (qPCR) (Clark et al., Hum. Gene Ther. 10, 1031-1039 (1999)) or dot-blot hybridization (Samulski et al., J. Virol. 63, 3822-3828 (1989)), or by optical density of highly purified vector preparations (Sommer et al., Mol. Ther. 7, 122-128 (2003)). DNase-resistant particles (DRP) can be quantified by real-time quantitative polymerase chain reaction (qPCR) (DRP-qPCR) in a thermocycler (for example, an iCycler iQ 96-well block format thermocycler (Bio-Rad, Hercules, Calif.)). Samples containing rAAV particles are incubated in the presence of DNase I (100 U/ml; Promega, Madison, Wis.) at 37° C. for 60 min, followed by proteinase K (Invitrogen, Carlsbad, Calif.) digestion (10 U/mL) at 50° C. for 60 min, and then denatured at 95° C. for 30 min. The primer-probe set used should be specific to a non-native portion of the rAAV vector genome, for example, the poly(A) sequence of the protein of interest. The PCR product can be amplified using any appropriate set of cycling parameters, based on the length and composition of the primers, probe, and amplified sequence. Alternative protocols are disclosed in, for example, Lock et al., Human Gene Therapy Methods 25(2): 115-125 (2014).

Throughout the description, where apparatus, devices, and systems are described as having, including, or comprising specific components, or where processes and methods are described as having, including, or comprising specific steps, it is contemplated that, additionally, there are apparatus, devices, and systems of the present invention that consist essentially of, or consist of, the recited components, and that there are processes and methods according to the present invention that consist essentially of, or consist of, the recited processing steps.

Practice of the invention will be more fully understood from the foregoing examples, which are presented herein for illustrative purposes only, and should not be construed as limiting the invention in any way.

### Examples

### Example 1: Effect of Different Concentrations of Niacinamide on rAAV Production with HeLa Cells

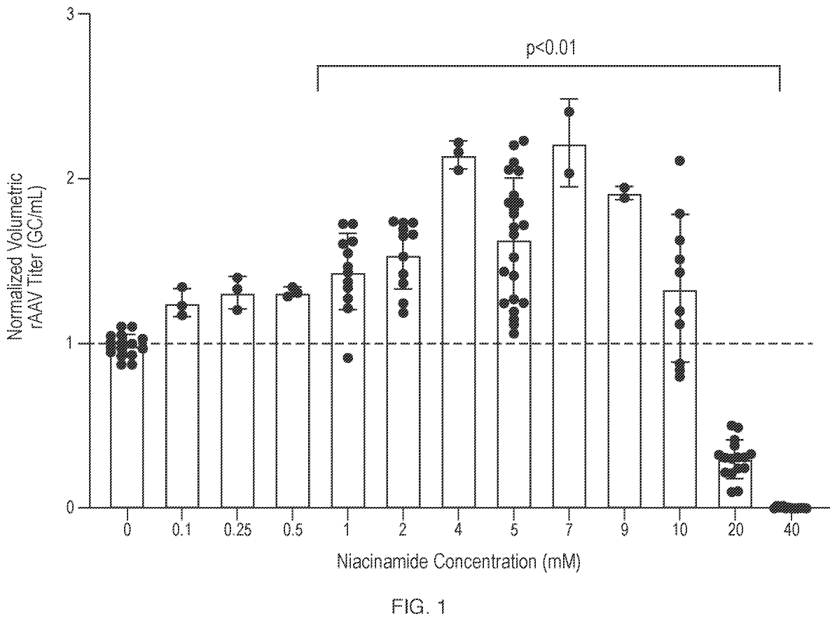

In this example, the effect of various concentrations of niacinamide ranging from 0.1 mM to 40 mM on rAAV production was tested in a variety HeLa producer cell lines. The experiment was run in a 24 deep-well plate culture system with a 3 mL working volume, a seeding density of 0.2×10⁶ cells in cell culture production medium, and an Ad5 multiplicity of infection (MOI) of 200 vp/cell. FIG. 1 shows a summary graph of normalized volumetric rAAV titer for a variety of Clade E and F rAAV produced from HeLa producer cells at varying niacinamide concentrations (0.1 mM-40 mM). Increases in rAAV titer were seen from 0.1 mM to 10 mM, with the most significant increases observed between 1 mM and 10 mM. The capsid serotypes included in this analysis were AAV8, AAV9, rh10, and hu37.

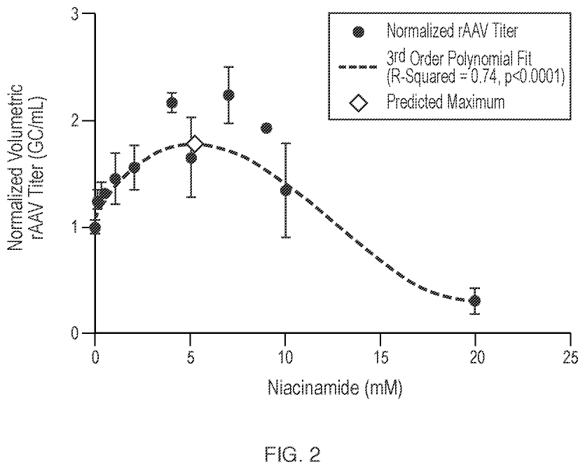

FIG. 2 shows a graph of normalized volumetric rAAV titer (GC/mL) per varying niacinamide concentrations as well as a predicted maximum when using a 3^{rd}-order polynomial fit of the data points. Significant increases in rAAV titer were seen between 1 mM and 10 mM, with a predicted maximum at 5 mM.

### Example 2: Effect of Vessel Scale on rAAV Production

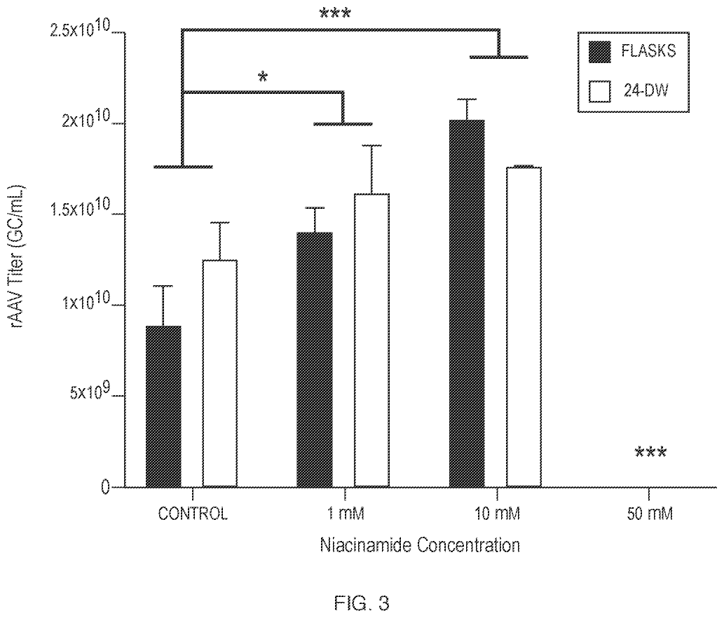

In this example, the effect of scale using two common vessels (3 mL-deep well (“24-DW”) and 100 mL shake flask (“Flasks”)) with niacinamide on rAAV production was tested. The analysis was conducted using a HeLa producer cell line capable of producing a Clade E rAAV encoding a Factor IX transgene (AAVrh10-FIX). FIG. 3 depicts a bar graph showing rAAV titer (GC/mL) at varying niacinamide concentrations at the different scales. A two-way ANOVA looking at the factors of niacinamide, scale, and the interaction between them showed a statistically significant impact only from niacinamide (p<0.0001). The scale and interaction factors were insignificant (p=0.3915 and p=0.1353 respectively). A Dunnett's Test was performed that showed significance at all concentrations when scale is removed as a factor.

### Example 3: Effect of Niacinamide on rAAV Production with Clade E HeLa Clones

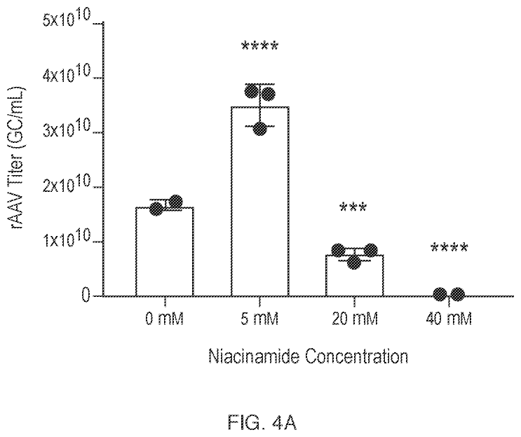

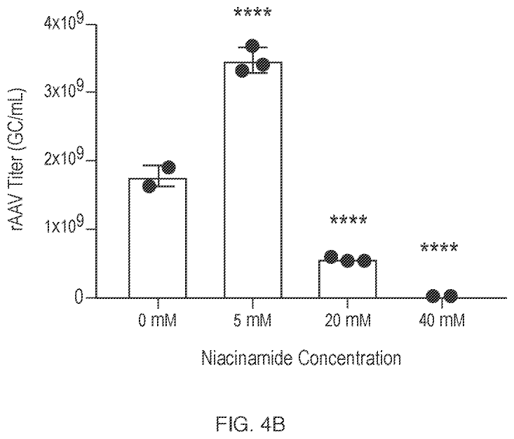

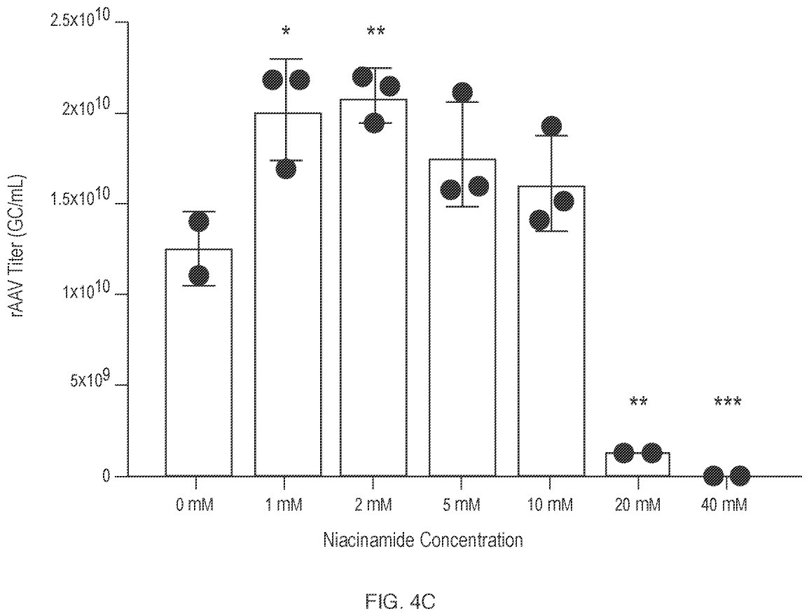

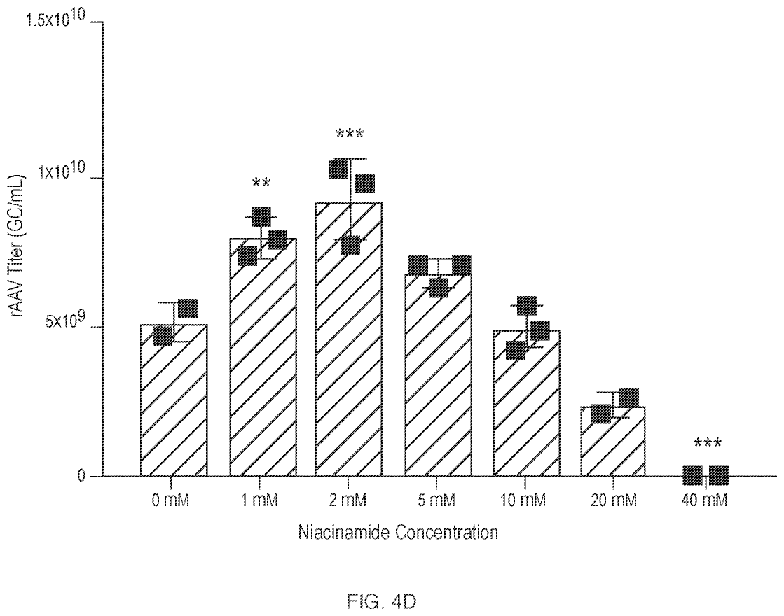

Four different HeLa clones were tested using the conditions described in Example 1. FIG. 4A-D show the rAAV titer (GC/mL) per varying niacinamide concentrations for Clone 1 (AAVrh10-FIX) follow-up supernatant, clone 2 (AAV8-G6Pase) supernatant, Clone 3 (AAV8-G6Pase) wide range with no supplement, and Clone 4 (AAVhu37-FVIII) wide range no supplement suspension (i.e., Triton-lysed sample), respectively. A titer increase was shown in all four clones when between 1 mM and 5 mM niacinamide was added.

### Example 4: Effect of Niacinamide on rAAV Production with Clade F HeLa Clones

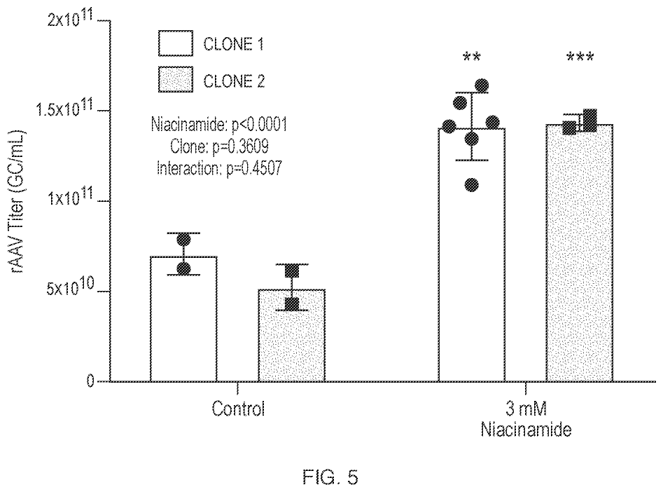

In this example, the effect of 3 mM niacinamide on two HeLa producer cell line clones expressing recombinant Clade F AAV encoding a truncated ATP7B transgene (AAV9-ATP7B) was examined in a 2 L bioreactor. As shown in FIG. 5, a significant titer increase was observed for both clones via the addition of 3 mM niacinamide to the bioreactor.

### Example 5: Effect of Different Concentrations of Niacinamide on rAAV Production with HEK293 Cells

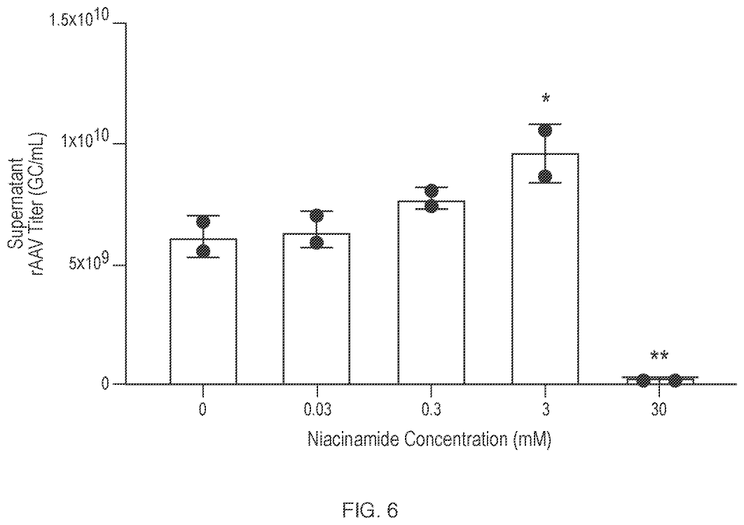

In this example, the effect of various concentrations of niacinamide ranging from 0.03 mM to 30 mM on rAAV production was tested with HEK293 cells. The experiment was run in 30 mL flasks using a transfection density of 1.3×10⁶ to 2.0×10⁶ cells/mL. The HEK293 cells used in this experiment were transfected with plasmids enabling the production of rAAV8 encoding an ornithine transcarbamylase (rAAV8-OTC). As shown in FIG. 6, increases in titer were observed with HEK293 cells at 0.3 mM and 3 mM niacinamide addition. A run repeated at a 2 L bioreactor scale also showed an increase in titer for HEK293 cells with addition of niacinamide (data not shown).

### Example 6: Effect of Additional Compounds on rAAV Production with HEK293 Cells

In this example, the effect of compounds other than niacinamide on rAAV production in HEK293 cells was examined. The compounds analyzed in this example included niacin, methyl nicotinate, myo-inositol, pantothenic acid, pyridoxine, choline, thiamine, glucosamine, caffeine, n-acetyl glucosamine, and thymidine.

The experiment was run in 24 deep-well plates using the same cells as described in Example 5 under similar transfection and culture conditions. The concentrations of each compound tested were 1 mM, 3 mM, and 9 mM. Amongst the compounds tested, niacin provided a 20% increase in titer at 1 mM, methyl nicotinate provided a 42% increase in titer at 3 mM, myo-inositol provided an approximately 20% increase in titer at 3 mM and 9 mM, while choline provided a 65% increase in titer at 3 mM. None of the other compounds tested provided a significant benefit to rAAV titer at any of the three concentrations tested.

This example shows that alternative compounds including niacin, methyl nicotinate, myo-inositol, and choline can boost rAAV titers, although none of the compounds provided the level of benefit provided by niacinamide which appears to be substantially and surprisingly superior to the other 11 compounds examined.

### NUMBERED EMBODIMENTS

Embodiments disclosed herein include embodiments P1 to P68 as provided in the numbered embodiments of the disclosure.

Embodiment P1: A method of producing recombinant adeno-associated virus (rAAV), the method comprising contacting a host cell with a solution comprising a compound of formula (I) or a salt thereof:

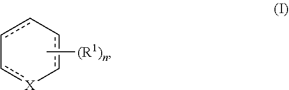

wherein:
[symbol] denotes a single bond or a double bond;
n is an integer selected from 1 to 5;
R¹ is selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b};
R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and wherein the compound is not:

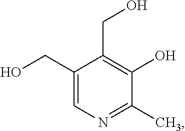 [SMILES: Cc1ncc(CO)c(CO)c1O]

or a salt thereof.

Embodiment P2: A method of increasing rAAV titer yield, the method comprising contacting a host cell with a solution comprising a compound of formula (I) or a salt thereof:

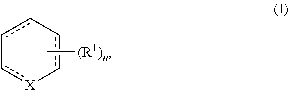

wherein:
[symbol] denotes a single bond or a double bond;
n is an integer selected from 1 to 5;
R¹ is selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR;
R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and
wherein the compound is not:

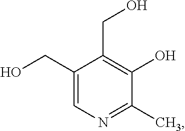 [SMILES: Cc1ncc(CO)c(CO)c1O]

or a salt thereof.

Embodiment P3: The method of embodiment P1 or P2, wherein X is N.

Embodiment P4: The method of any one of embodiments P1-P3, wherein n is 1.

Embodiment P5: The method of any one of embodiments P1-P4, wherein R¹ is selected from the group consisting of C₁₋₆alkyl-OH, —C(O)N(R^{a})₂, and —C(O)OR^{b}.

Embodiment P6: The method of any one of embodiments P1-P5, wherein R¹ is selected from the group consisting of CH₂OH, —C(O)NH₂, —C(O)OH, and —C(O)OCH₃.

Embodiment P7: The method of embodiment P1 or P2, wherein X is CR^{c}R^{d}.

Embodiment P8: The method of any one of embodiments P1, P2, and P7, wherein R^{c} is hydrogen and R^{d} is —OH.

Embodiment P9: The method of any one of embodiments P1, P2, and P7-P8, wherein n is 5.

Embodiment P10: The method of any one of embodiments P1, P2, and P7-P9, wherein R¹ is —OH.

Embodiment P11: The method of embodiment P1 or P2, wherein the compound is a compound of formula (I-A) or a salt thereof:

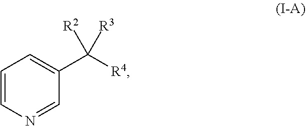

wherein:
R² and R³ are independently, for each occurrence, hydrogen or —OH,
or R² and R³ can be taken together to form oxo;
R⁴ is selected from the group consisting of hydrogen, —OH, —O—C₁₋₆alkyl, and —N(R^{e})₂; and
R^{e} is independently, for each occurrence, hydrogen or C₁₋₆alkyl.

Embodiment P12: The method of embodiment P11, wherein R² and R³ are taken together to form oxo.

Embodiment P13: The method of embodiment P11 or P12, wherein R⁴ is selected from the group consisting of —NH₂, —OH, and —OCH₃.

Embodiment P14: The method of embodiment P11, wherein R² and R³ are both hydrogen.

Embodiment P15: The method of embodiment P11 or P14, wherein R⁴ is —OH.

Embodiment P16: The method of any one of embodiments P1, P2, and P11, wherein the compound is selected from the group consisting of:

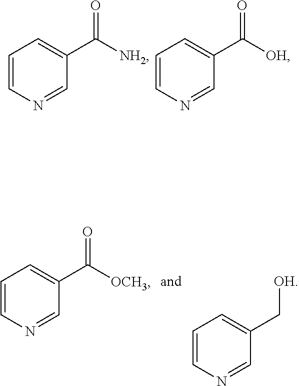

Embodiment P17: The method of embodiment P1 or P2, wherein the compound is a compound of formula (I-B) or a salt thereof:

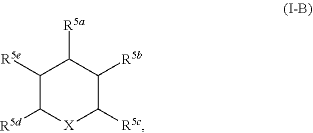

wherein:
X is CR^{c}R^{d}; and
R^{5a}, R^{5b}, R^{5c}, R^{5d}, and R^{5e} are independently, for each occurrence, —OH, —O—C₁₋₆alkyl, C₁₋₆alkyl, C₁₋₆alkyl-OH, and C₁₋₆alkyl-O—C₁₋₆alkyl, wherein R^{c} and R^{d} are as defined in embodiment P1.

Embodiment P18: The method of embodiment P17, wherein R^{c} is hydrogen and R^{d} is —OH.

Embodiment P19: The method of embodiment P17 or P18, wherein R^{5a}, R^{5b}, R^{5c}, R^{5d}, and R^{5e} are —OH.

Embodiment P20: The method of any one of embodiments P17-P19, wherein the compound is

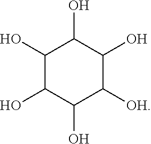 [SMILES: OC1C(O)C(O)C(O)C(O)C1O]

Embodiment P21: A method of producing rAAV, the method comprising contacting a host cell with a solution comprising a compound of formula (II) or a salt thereof:

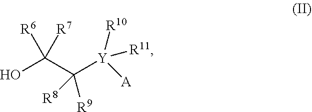

wherein:
R⁶ and R⁷ are independently, for each occurrence, selected from the group consisting of hydrogen, —OH, —CN, halogen, and C₁₋₆alkyl;
R⁸ and R⁹ are independently, for each occurrence, selected from the group consisting of hydrogen, —OH, —CN, halogen, and C₁₋₆alkyl;
Y is C or N;
R¹⁰ and R¹¹ are independently, for each occurrence, selected from the group consisting of hydrogen, —OH, and C₁₋₆alkyl;
A is selected from the group consisting of hydrogen, C₁₋₆alkyl, and —C(O)N(R^{f})₂; and
R^{f} is independently, for each occurrence, selected from the group consisting of hydrogen, C₁₋₆alkyl, C₁₋₆alkyl-C(O)OH, and C₁₋₆alkyl-OH;
wherein the compound is not:

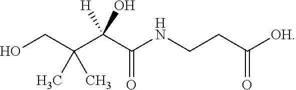

Embodiment P22: A method of increasing rAAV titer yield, the method comprising contacting a host cell with a solution comprising a compound of formula (II) or a salt thereof:

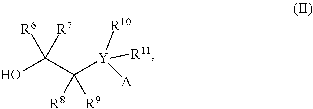

wherein:
R⁶ and R⁷ are independently, for each occurrence, selected from the group consisting of hydrogen, —OH, —CN, halogen, and C₁₋₆alkyl;
R⁸ and R⁹ are independently, for each occurrence, selected from the group consisting of hydrogen, —OH, —CN, halogen, and C₁₋₆alkyl;
Y is C or N;
R¹⁰ and R¹¹ are independently, for each occurrence, selected from the group consisting of hydrogen, —OH, and C₁₋₆alkyl;
A is selected from the group consisting of hydrogen, C₁₋₆alkyl, and —C(O)N(R^{f})₂; and
R^{f} is independently, for each occurrence, selected from the group consisting of hydrogen, C₁₋₆alkyl, C₁₋₆alkyl-C(O)OH, and C₁₋₆alkyl-OH;
wherein the compound is not:

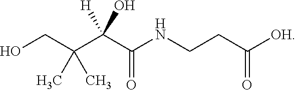

Embodiment P23: The method of embodiment P21 or P22, wherein R⁶ and R⁷ are hydrogen.

Embodiment P24: The method of any one of embodiments P21-P23, wherein R⁸ and R⁹ are hydrogen.

Embodiment P25: The method of any one of embodiments P21-P24, wherein Y is N.

Embodiment P26: The method of any one of embodiments P21-P25, wherein R¹⁰ and R¹¹ are CH₃.

Embodiment P27: The method of any one of embodiments P21-P26, wherein A is CH₃.

Embodiment P28: The method of any one of embodiments P21-P27, wherein the compound is

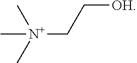

Embodiment P29: A method of producing rAAV or increasing rAAV titer yield, the method comprising contacting a host cell with a solution comprising a B vitamin selected from the group consisting of vitamin B₂, vitamin B₇, vitamin B₉, and vitamin B₁₂.

Embodiment P30: The method of any one of embodiments P1-P29, wherein the final concentration of the compound in the solution is greater than 0.5 mM.

Embodiment P31: The method of any one of embodiments P1-P29, wherein the final concentration of the compound in the solution is greater than or equal to 0.5 mM.

Embodiment P32: The method of any one of embodiments P1-P29, wherein the final concentration of the compound in the solution is between 0.5 mM and 10 mM.

Embodiment P33: The method of any one of embodiments P1-P29, wherein the final concentration of the compound in the solution is between 1 mM and 10 mM.

Embodiment P34: The method of any one of embodiments P1-P33, wherein the final concentration of the compound in the solution is sufficient to produce at least 1.2-fold greater quantities of secreted rAAV compared to that produced by a host cell not contacted with a solution comprising the compound.

Embodiment P35: The method of any one of embodiments P1-P33, wherein the final concentration of the compound in the solution is sufficient to produce at least 1.2-fold greater quantities of total rAAV compared to that produced by a host cell not contacted with a solution comprising the compound.

Embodiment P36: The method of any one of embodiments P1-P33, wherein the final concentration of the compound in the solution is sufficient to produce 1.2 to 2.5-fold greater quantities of secreted rAAV compared to that produced by a host cell not contacted with a solution comprising the compound.

Embodiment P37: The method of any one of embodiments P1-P33, wherein the final concentration of the compound in the solution is sufficient to produce 1.2 to 2.5-fold greater quantities of total rAAV compared to that produced by a host cell not contacted with a solution comprising the compound.

Embodiment P38: The method of any one of embodiments P1-P37, wherein the host cell is contacted with the solution comprising the compound for at least 2 days.

Embodiment P39: The method of any one of embodiments P1-P38, further comprising the steps of harvesting and purifying the rAAV.

Embodiment P40: The method of any one of embodiments P1-P39, wherein the host cell is a mammalian cell.

Embodiment P41: The method of embodiment P40, wherein the host cell is selected from the group consisting of HeLa, HEK293, COS, A549, BHK, and Vero cells.

Embodiment P42: The method of embodiment P41, wherein the host cell is a HeLa cell.

Embodiment P43: The method of embodiment P41, wherein the host cell is a HEK293 cell.

Embodiment P44: The method of any one of embodiments P1-P39, wherein the host cell is an insect cell.

Embodiment P45: The method of embodiment P44, wherein the host cell is selected from the group consisting of Sf, Sf-21, Tn-368, and BTI-Tn-5B1-4 (High-Five) cells.

Embodiment P46: The method of any one of embodiments P1-P45, wherein the host cell comprises a heterologous nucleotide sequence flanked by AAV inverted terminal repeats.

Embodiment P47: The method of any one of embodiments P1-P46, wherein the host cell comprises rep and cap genes.

Embodiment P48: The method of any one of embodiments P1-P47, wherein the host cell comprises helper virus genes.

Embodiment P49: The method of any one of embodiments P1-P48, wherein the host cell comprises a heterologous nucleotide sequence flanked by AAV inverted terminal repeats, rep and cap genes, and helper virus genes.

Embodiment P50: The method of any one of embodiments P1-P49, wherein the host cell produces at least 1.2-fold greater quantities of secreted rAAV compared to that produced by a host cell not contacted with a solution comprising the compound.

Embodiment P51: The method of any one of embodiments P1-P49, wherein the host cell produces at least 1.2-fold greater quantities of total rAAV compared to that produced by a host cell not contacted with a solution comprising the compound.

Embodiment P52: The method of any one of embodiments P1-P49, wherein the host cell produces 1.2 to 2.5-fold greater quantities of secreted rAAV compared to that produced by a host cell not contacted with a solution comprising the compound.

Embodiment P53: The method of any one of embodiments P1-P49, wherein the host cell produces 1.2 to 2.5-fold greater quantities of total rAAV compared to that produced by a host cell not contacted with a solution comprising the compound.

Embodiment P54: The method of any one of embodiments P1-P53, wherein the solution comprises cell culture medium.

Embodiment P55: The method of any one of embodiments P1-P53, wherein the solution comprises production medium.

Embodiment P56: The method of any one of embodiments P1-P55 comprising culturing the host cell in a suspension culture.

Embodiment P57: The method of any one of embodiments P1-P55 comprising culturing the host cell in an adherent culture.

Embodiment P58: The method of any one of embodiments P1-P56 comprising culturing the host cell in a 1 L bioreactor.

Embodiment P59: The method of any one of embodiments P1-P56 comprising culturing the host cell in a 2 L bioreactor.

Embodiment P60: The method of any one of embodiments P1-56 comprising culturing the host cell in a 3 L bioreactor.

Embodiment P61: The method of any one of embodiments P1-P56 comprising culturing the host cell in a 250 L bioreactor.

Embodiment P62: The method of any one of embodiments P1-P56 comprising culturing the host cell in a 500 L bioreactor.

Embodiment P63: The method of any one of embodiments P1-P56 comprising culturing the host cell in a 2,000 L bioreactor.

Embodiment P64: A composition comprising a host cell and a compound of formula (I) or a salt thereof.

Embodiment P65: A composition comprising a host cell and a compound of formula (I-A) or a salt thereof.

Embodiment P66: A composition comprising a host cell and a compound of formula (I-B) or a salt thereof.

Embodiment P67: A composition comprising a host cell and a compound of formula (II) or a salt thereof.

Embodiment P68: A composition comprising a host cell and a B vitamin selected from the group consisting of vitamin B₂, vitamin B₇, vitamin B₉, and vitamin B₁₂.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of producing recombinant adeno-associated virus (rAAV), the method comprising contacting a host cell with a solution comprising at least one compound of formula (I) or a salt thereof: 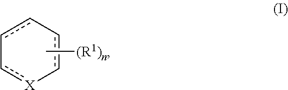
wherein: [symbol]
denotes a single bond or a double bond;
n is an integer selected from 1 to 5;
each R¹ is independently, for each occurrence, selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b};
R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and
wherein the at least one compound is not: 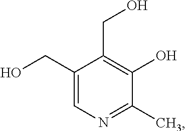 [SMILES: Cc1ncc(CO)c(CO)c1O]
or a salt thereof.

2. A method of increasing rAAV titer yield, the method comprising contacting a host cell with a solution comprising at least one compound of formula (I) or a salt thereof: 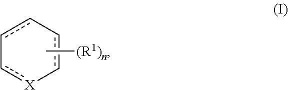
wherein: [symbol]
denotes a single bond or a double bond;
n is an integer selected from 1 to 5;
each R¹ is independently, for each occurrence, selected from the group consisting of —OH, C₁₋₆alkyl, C₁₋₆alkyl-OH, C₁₋₆alkyl-O—C₁₋₆alkyl, —O—C₁₋₆alkyl, —C(O)N(R^{a})₂, and —C(O)OR^{b};
R^{a} is independently, for each occurrence, hydrogen or C₁₋₆alkyl;
R^{b} is independently, for each occurrence, hydrogen or C₁₋₆alkyl;
X is CR^{c}R^{d} or N;
R^{c} and R^{d} are independently, for each occurrence, selected from the group consisting of hydrogen, C₁₋₆alkyl, —OH, and —O—C₁₋₆alkyl, wherein when R^{c} is hydrogen, R^{d} is selected from C₁₋₆alkyl, —OH, and —O—C₁₋₆alkyl, and when R^{c} is selected from C₁₋₆alkyl, —OH, and —O—C₁₋₆alkyl, R^{d} is hydrogen; and
wherein the at least one compound is not: 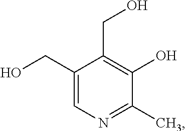 [SMILES: Cc1ncc(CO)c(CO)c1O]
or a salt thereof.

3. The method of claim 1 or 2, wherein X is N.

4. The method of any one of claims 1-3, wherein n is 1.

5. The method of any one of claims 1-4, wherein each R¹ is independently, for each occurrence, selected from the group consisting of C₁₋₆alkyl-OH, —C(O)N(R^{a})₂, and —C(O)OR^{b}.

6. The method of any one of claims 1-5, wherein each R¹ is independently, for each occurrence, selected from the group consisting of CH₂OH, —C(O)NH₂, —C(O)OH, and —C(O)OCH₃.

7. The method of claim 1 or 2, wherein X is CR^{c}R^{d}.

8. The method of any one of claims 1, 2, and 7, wherein R^{c} is hydrogen and R^{d} is —OH.

9. The method of any one of claims 1, 2, 3, and 7-8, wherein n is 5.

10. The method of any one of claims 1, 2, 3, 4, and 7-9, wherein R¹ is —OH.

11. The method of claim 1 or 2, wherein the solution comprises at least one compound of formula (I-A) or a salt thereof: 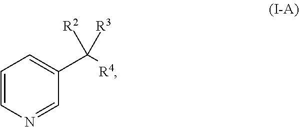
wherein:
R² and R³ are independently, for each occurrence, hydrogen or —OH,
or R² and R³ can be taken together to form oxo;
R⁴ is selected from the group consisting of hydrogen, —OH, —O—C₁₋₆alkyl, and —N(R^{e})₂; and
R^{e} is independently, for each occurrence, hydrogen or C₁₋₆alkyl.

12. The method of claim 11, wherein R² and R³ are taken together to form oxo.

13. The method of claim 11 or 12, wherein R⁴ is selected from the group consisting of —NH₂, —OH, and —OCH₃.

14. The method of claim 11, wherein R² and R³ are both hydrogen.

15. The method of claim 11 or 14, wherein R⁴ is —OH.

16. The method of any one of claims 1, 2, and 11, wherein the solution comprises at least one compound selected from the group consisting of: 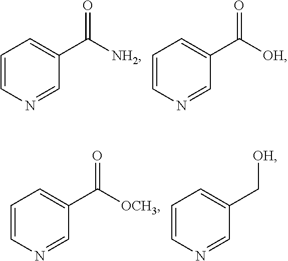
and any combination(s) thereof.

17. The method of claim 1 or 2, wherein the solution comprises at least one compound of formula (I-B) or a salt thereof: 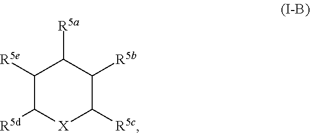
wherein:
X is CR^{c}R^{d}; and
R^{5a}, R^{5b}, R^{5c}, R^{5d}, and R^{5e} are independently, for each occurrence, —OH, —O—C₁₋₆alkyl, C₁₋₆alkyl, C₁₋₆alkyl-OH, and C₁₋₆alkyl-O—C₁₋₆alkyl,
wherein R^{c} and R^{d} are as defined in claim 1.

18. The method of claim 17, wherein R^{c} is hydrogen and R^{d} is —OH.

19. The method of claim 17 or 18, wherein R^{5a}, R^{5b}, R^{5c}, R^{5d}, and R^{5e} are —OH.

20. The method of any one of claims 17-19, wherein the solution comprises the compound

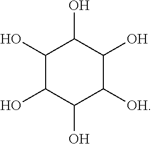 [SMILES: OC1C(O)C(O)C(O)C(O)C1O]

21. A method of producing rAAV, the method comprising contacting a host cell with a solution comprising at least one compound of formula (II) or a salt thereof: 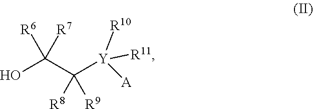
wherein:
R⁶ and R⁷ are independently, for each occurrence, selected from the group consisting of hydrogen, —OH, —CN, halogen, and C₁₋₆alkyl;
R⁸ and R⁹ are independently, for each occurrence, selected from the group consisting of hydrogen, —OH, —CN, halogen, and C₁₋₆alkyl;
Y is C or N;
R¹⁰ and R¹¹ are independently, for each occurrence, selected from the group consisting of hydrogen, —OH, and C₁₋₆alkyl;
A is selected from the group consisting of hydrogen, C₁₋₆alkyl, and —C(O)N(R^{f})₂; and
R^{f} is independently, for each occurrence, selected from the group consisting of hydrogen, C₁₋₆alkyl, C₁₋₆alkyl-C(O)OH, and C₁₋₆alkyl-OH;
wherein the at least one compound is not: 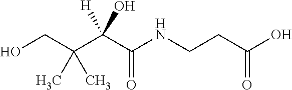
or a salt thereof.

22. A method of increasing rAAV titer yield, the method comprising contacting a host cell with a solution comprising at least one compound of formula (II) or a salt thereof: 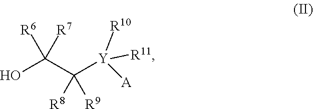
wherein:
R⁶ and R⁷ are independently, for each occurrence, selected from the group consisting of hydrogen, —OH, —CN, halogen, and C₁₋₆alkyl;
R⁸ and R⁹ are independently, for each occurrence, selected from the group consisting of hydrogen, —OH, —CN, halogen, and C₁₋₆alkyl;
Y is C or N;
R¹⁰ and R¹¹ are independently, for each occurrence, selected from the group consisting of hydrogen, —OH, and C₁₋₆alkyl;
A is selected from the group consisting of hydrogen, C₁₋₆alkyl, and —C(O)N(R^{f})₂; and
R^{f} is independently, for each occurrence, selected from the group consisting of hydrogen, C₁₋₆alkyl, C₁₋₆alkyl-C(O)OH, and C₁₋₆alkyl-OH;
wherein the at least one compound is not: 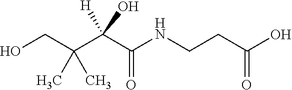
or a salt thereof.

23. The method of claim 21 or 22, wherein R⁶ and R⁷ are hydrogen.

24. The method of any one of claims 21-23, wherein R⁸ and R⁹ are hydrogen.

25. The method of any one of claims 21-24, wherein Y is N.

26. The method of any one of claims 21-25, wherein R¹⁰ and R¹¹ are CH₃.

27. The method of any one of claims 21-26, wherein A is CH₃.

28. The method of any one of claims 21-27, wherein the solution comprises the compound

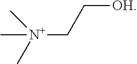

29. A method of producing rAAV or increasing rAAV titer yield, the method comprising contacting a host cell with a solution comprising at least one B vitamin selected from the group consisting of vitamin B₂, vitamin B₇, vitamin B₉, vitamin B₁₂, and any combination(s) thereof.

30. The method of any one of claims 1-29, wherein the final concentration of the at least one compound or B vitamin in the solution is greater than 0.5 mM.

31. The method of any one of claims 1-29, wherein the final concentration of the at least one compound or B vitamin in the solution is greater than or equal to 0.5 mM.

32. The method of any one of claims 1-29, wherein the final concentration of the at least one compound or B vitamin in the solution is between 0.5 mM and 10 mM.

33. The method of any one of claims 1-29, wherein the final concentration of the at least one compound or B vitamin in the solution is between 1 mM and 10 mM.

34. The method of any one of claims 1-33, wherein the final concentration of the at least one compound or B vitamin in the solution is sufficient to produce at least 1.2-fold greater quantities of secreted rAAV compared to that produced by a host cell not contacted with a solution comprising the at least one compound or B vitamin.

35. The method of any one of claims 1-33, wherein the final concentration of the at least one compound in the solution is sufficient to produce at least 1.2-fold greater quantities of total rAAV compared to that produced by a host cell not contacted with a solution comprising the at least one compound or B vitamin.

36. The method of any one of claims 1-33, wherein the final concentration of the at least one compound in the solution is sufficient to produce 1.2 to 2.5-fold greater quantities of secreted rAAV compared to that produced by a host cell not contacted with a solution comprising the at least one compound or B vitamin.

37. The method of any one of claims 1-33, wherein the final concentration of the at least one compound in the solution is sufficient to produce 1.2 to 2.5-fold greater quantities of total rAAV compared to that produced by a host cell not contacted with a solution comprising the at least one compound or B vitamin.

38. The method of any one of claims 1-37, wherein the host cell is contacted with the solution comprising the at least one compound or B vitamin for at least 2 days.

39. The method of any one of claims 1-38, further comprising the steps of harvesting and purifying the rAAV.

40. The method of any one of claims 1-39, wherein the host cell is a mammalian cell.

41. The method of claim 40, wherein the host cell is selected from the group consisting of HeLa, HEK293, COS, A549, BHK, and Vero cells.

42. The method of claim 41, wherein the host cell is a HeLa cell.

43. The method of claim 41, wherein the host cell is a HEK293 cell.

44. The method of any one of claims 1-39, wherein the host cell is an insect cell.

45. The method of claim 44, wherein the host cell is selected from the group consisting of Sf9, Sf-21, Tn-368, and BTI-Tn-5B1-4 (High-Five) cells.

46. The method of any one of claims 1-45, wherein the host cell comprises a heterologous nucleotide sequence flanked by AAV inverted terminal repeats.

47. The method of any one of claims 1-46, wherein the host cell comprises rep and cap genes.

48. The method of any one of claims 1-47, wherein the host cell comprises helper virus genes.

49. The method of any one of claims 1-48, wherein the host cell comprises i) a heterologous nucleotide sequence flanked by AAV inverted terminal repeats, ii) rep and cap genes, and iii) helper virus genes.

50. The method of any one of claims 1-49, wherein the host cell produces at least 1.2-fold greater quantities of secreted rAAV compared to that produced by a host cell not contacted with a solution comprising the at least one compound.

51. The method of any one of claims 1-49, wherein the host cell produces at least 1.2-fold greater quantities of total rAAV compared to that produced by a host cell not contacted with a solution comprising the at least one compound or B vitamin.

52. The method of any one of claims 1-49, wherein the host cell produces 1.2 to 2.5-fold greater quantities of secreted rAAV compared to that produced by a host cell not contacted with a solution comprising the at least one compound or B vitamin.

53. The method of any one of claims 1-49, wherein the host cell produces 1.2 to 2.5-fold greater quantities of total rAAV compared to that produced by a host cell not contacted with a solution comprising the at least one compound or B vitamin.

54. The method of any one of claims 1-53, wherein the solution comprises cell culture medium.

55. The method of any one of claims 1-53, wherein the solution comprises production medium.

56. The method of any one of claims 1-55 comprising culturing the host cell in a suspension culture.

57. The method of any one of claims 1-55 comprising culturing the host cell in an adherent culture.

58. The method of any one of claims 1-56 comprising culturing the host cell in a 1 L bioreactor.

59. The method of any one of claims 1-56 comprising culturing the host cell in a 2 L bioreactor.

60. The method of any one of claims 1-56 comprising culturing the host cell in a 3 L bioreactor.

61. The method of any one of claims 1-56 comprising culturing the host cell in a 250 L bioreactor.

62. The method of any one of claims 1-56 comprising culturing the host cell in a 500 L bioreactor.

63. The method of any one of claims 1-56 comprising culturing the host cell in a 2,000 L bioreactor.

64. A composition comprising a host cell and a compound of formula (I) or a salt thereof.

65. A composition comprising a host cell and a compound of formula (I-A) or a salt thereof.

66. A composition comprising a host cell and a compound of formula (I-B) or a salt thereof.

67. A composition comprising a host cell and a compound of formula (II) or a salt thereof.

68. A composition comprising a host cell and at least one B vitamin selected from the group consisting of vitamin B₂, vitamin B₇, vitamin B₉, and vitamin B₁₂, and any combination(s) thereof.

69. A method of producing recombinant adeno-associated virus (rAAV), the method comprising contacting a host cell with a solution comprising at least one compound selected from the group consisting of: 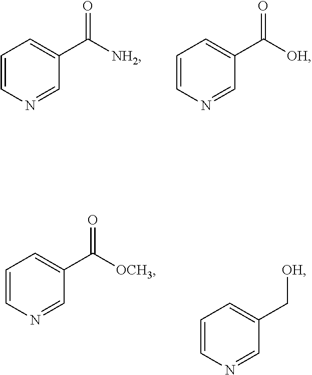 [SMILES: NC(=O)c1cccnc1 ; O=C(O)c1cccnc1 ; COC(=O)c1cccnc1 ; OCc1cccnc1]
and any combination(s) thereof
wherein the host cell comprises:
i) a heterologous nucleotide sequence flanked by AAV inverted terminal repeats, and
ii) AAV rep and cap genes.

70. A method of increasing rAAV titer yield, the method comprising contacting a host cell with a solution comprising at least one compound selected from the group consisting of: 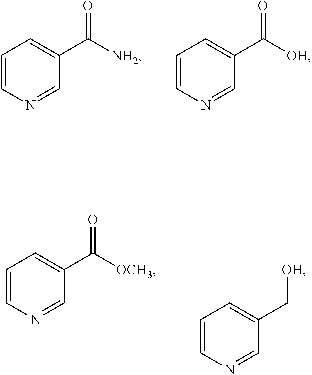 [SMILES: NC(=O)c1cccnc1 ; O=C(O)c1cccnc1 ; COC(=O)c1cccnc1 ; OCc1cccnc1]
and any combination(s) thereof
wherein the host cell comprises:
i) a heterologous nucleotide sequence flanked by AAV inverted terminal repeats, and
ii) AAV rep and cap genes.

71. A composition comprising a host cell and at least one compound selected from the group consisting of: 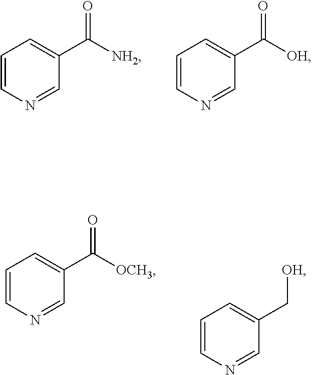 [SMILES: NC(=O)c1cccnc1 ; O=C(O)c1cccnc1 ; COC(=O)c1cccnc1 ; OCc1cccnc1]
and any combination(s) thereof
wherein the host cell comprises:
i) a heterologous nucleotide sequence flanked by AAV inverted terminal repeats, and
ii) AAV rep and cap genes.

72. A method of producing recombinant adeno-associated virus (rAAV), the method comprising contacting a host cell with a solution comprising 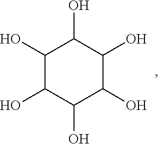 [SMILES: OC1C(O)C(O)C(O)C(O)C1O]
wherein the host cell comprises:
i) a heterologous nucleotide sequence flanked by AAV inverted terminal repeats, and
ii) AAV rep and cap genes.

73. A method of increasing rAAV titer yield, the method comprising contacting a host cell with a solution comprising 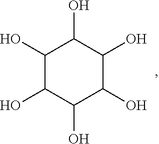 [SMILES: OC1C(O)C(O)C(O)C(O)C1O]
wherein the host cell comprises:
i) a heterologous nucleotide sequence flanked by AAV inverted terminal repeats, and
ii) AAV rep and cap genes.

74. A composition comprising a host cell and 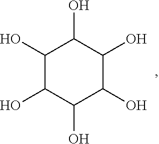 [SMILES: OC1C(O)C(O)C(O)C(O)C1O]
wherein the host cell comprises:
i) a heterologous nucleotide sequence flanked by AAV inverted terminal repeats, and
ii) AAV rep and cap genes.

75. A method of producing recombinant adeno-associated virus (rAAV), the method comprising contacting a host cell with a solution comprising: 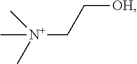
wherein the host cell comprises:
i) a heterologous nucleotide sequence flanked by AAV inverted terminal repeats, and
ii) AAV rep and cap genes.

76. A method of increasing rAAV titer yield, the method comprising contacting a host cell with a solution comprising: 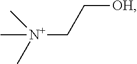
wherein the host cell comprises:
i) a heterologous nucleotide sequence flanked by AAV inverted terminal repeats, and
ii) AAV rep and cap genes.

77. A composition comprising a host cell and 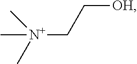
wherein the host cell comprises:
i) a heterologous nucleotide sequence flanked by AAV inverted terminal repeats, and
ii) AAV rep and cap genes.

78. A method of producing recombinant adeno-associated virus (rAAV), the method comprising contacting a host cell with a solution comprising at least one B vitamin selected from the group consisting of vitamin B₂, vitamin B₇, vitamin B₉, and vitamin B₁₂, and any combination(s) thereof, wherein the host cell comprises:
i) a heterologous nucleotide sequence flanked by AAV inverted terminal repeats, and
ii) AAV rep and cap genes.

79. A method of increasing rAAV titer yield, the method comprising contacting a host cell with a solution comprising at least one B vitamin selected from the group consisting of vitamin B₂, vitamin B₇, vitamin B₉, and vitamin B₁₂, and any combination(s) thereof, wherein the host cell comprises:
i) a heterologous nucleotide sequence flanked by AAV inverted terminal repeats, and
ii) AAV rep and cap genes.

80. The method of any one of claims 69, 70, 72, 73, 75, 76, 78, or 79, wherein the host cell further comprises helper genes.

81. A composition comprising a host cell and at least one B vitamin selected from the group consisting of vitamin B₂, vitamin B₇, vitamin B₉, vitamin B₁₂, and any combination(s) thereof, wherein the host cell comprises:
i) a heterologous nucleotide sequence flanked by AAV inverted terminal repeats, and
ii) AAV rep and cap genes.

82. The composition of any one of claims 71, 74, 77, or 81, wherein the host cell further comprises helper genes.

83. The method of any one of claims 1-20, wherein the solution does not comprise 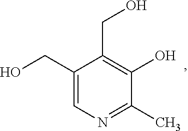
or a salt thereof.

84. The method of any one of claims 21-28, wherein the solution does not comprise 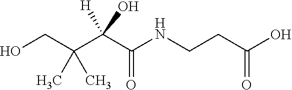
or a salt thereof.

85. The composition of any one of claims 64-66, 71, or 74, wherein the composition does not comprise 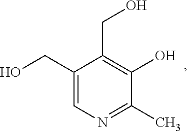
or a salt thereof.

86. The composition of claim 67 or 77, wherein the composition does not comprise 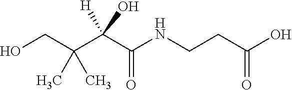
or a salt thereof.
